(19) United States
(12) Patent Application Publication
(10) Pub. No.: US 2012/0046333 A1
(43) Pub. Date: Feb. 23, 2012

(54) Methods and Compositions of PI-3 Kinase Inhibitors for Treating Fibrosis

(75) Inventors: William Hardie, Lebanon, OH (US); Robert Kirkman, Yarrow Point, WA (US)

(73) Assignee: Oncothyreon Incorporated, Seattle, WA (US)

(21) Appl. No.: 13/262,906

(22) PCT Filed: Apr. 8, 2010
(86) PCT No.: PCT/US10/30420

§ 371 Date: Nov. 7, 2011

Related U.S. Application Data
(60) Provisional application No. 61/167,905, filed on Apr. 9, 2009.
(60) Provisional application No. 61/235,740, filed on Aug. 21, 2009.

(51) Int. Cl. A61K 31/4025; A61P 11/00; A61K 31/352

(52) U.S. Cl. 514/422; 514/453

(57) ABSTRACT

Methods and compositions of PI-3 kinase inhibitors and their use in inhibiting PI-3 kinase activity in mammals and the treatment of fibrosing syndromes in a subject are described herein

### CROSS-REFERENCE

This patent application claims the benefit of U.S. Provisional Application Ser. No. 61/167,905 filed Apr. 9, 2009 and U.S. Provisional Application Ser. No. 61/235,740 filed Aug. 21, 2009; all of which are incorporated by reference herein in their entirety.

### BACKGROUND

Deposition of excess connective tissue during tissue reparative processes causes fibrosis. In some instances, fibrosis occurs when abnormal and/or excess fibrous connective tissue spreads over or replaces tissue lost due to, for example, injury, disease or infection.

### SUMMARY OF THE INVENTION

Provided herein are methods of treating fibrosing syndromes comprising administration of wortmannin or wortmannin analogs to individuals in need thereof. Also described herein are methods of treating pulmonary fibrosis comprising administration of PI-3 kinase (PI3K) inhibitors to individuals in need thereof. Further described herein are methods of treating pulmonary fibrosis comprising administration of wortmannin or wortmannin analogs to individuals in need thereof. In some instances, fibrosis is associated with development of abnormal fibrous connective tissue in an organ. In some instances, fibrosis causes scarring in the affected organ, thereby disrupting the functional and/or structural architecture of the underlying organ. In some instances, fibrosis occurs in an organ subsequent to organ transplant and/or organ allograft surgery. In some instances, activation of PI-3 kinases is associated with onset and/or progression of fibrosing syndromes as described herein.

Accordingly, described herein are methods of reducing or partially reducing activity of PI-3 kinases, thereby reversing fibrosis or delaying the progression of fibrosis or preventing the establishment of fibrosis (e.g., after organ transplant). In some embodiments, wortmannin analogs described herein are PI-3 kinase inhibitors. In some embodiments, PI 3 kinase inhibitors described herein are reversible PI-3 kinase inhibitors. In other embodiments, PI-3 kinase inhibitors described herein are irreversible PI-3 kinase inhibitors.

Provided herein, in some embodiments, are methods of treatment of mild or moderate or severe pulmonary fibrosis comprising administration of a PI-3 kinase inhibitor to an individual in need thereof.

In some embodiments, the PI-3 kinase inhibitor selectively inhibits PI-3 kinase alpha, PI-3 kinase beta, PI-3 kinase delta or PI-3 kinase gamma or a combination thereof. In some embodiments, the PI-3 kinase inhibitor selectively inhibits PI-3 kinase alpha or PI-3 kinase beta or a combination thereof.

In some embodiments, the PI-3 kinase inhibitor is a reversible inhibitor of a PI-3 kinase. In some embodiments, the PI-3 kinase inhibitor is an irreversible inhibitor of a PI-3 kinase.

In some embodiments, the pulmonary fibrosis is idiopathic pulmonary fibrosis. In some embodiments, the pulmonary fibrosis is associated with asbestosis, cystic fibrosis, infection (e.g., pneumonia), exposure to environmental allergens (e.g., coal dust, asbestos, cigarette smoke, diesel exhaust, ozone, particulates from industry emissions), lung transplant, autoimmune disease (e.g., scleroderma), or the pulmonary fibrosis is drug-induced pulmonary fibrosis.

In some embodiments of the methods described above, administration of a PI-3 kinase inhibitor reduces or reverses or decreases progression of lung fibrosis. In some embodiments of the methods described above, administration of a PI-3 kinase inhibitor prevents progressive weight loss. In some embodiments of the methods described above, administration of a PI-3 kinase inhibitor slows progression of TGF-alpha-dependent changes in lung mechanics. In some embodiments, administration of a PI-3 kinase inhibitor prevents establishment of pulmonary fibrosis. In some embodiments, a PI-3 kinase inhibitor is administered prophylactically (e.g., prior to a lung transplant). In some embodiments, a PI-3 kinase inhibitor is administered therapeutically (e.g., after onset of mild or moderate or severe plumonary fibrosis).

In some embodiments of the methods, one or more PI-3 kinase inhibitors are administered orally. In some embodiments of the methods, one or more PI-3 kinase inhibitors are administered as an inhalable formulation.

Also provided herein are methods of treating a fibrosing syndrome in an individual diagnosed with or suspected of having a fibrosing syndrome comprising administering to the individual in need thereof a therapeutically effective amount of a wortmannin analogue.

In some embodiments, the wortmannin analogue is a compound of formula:

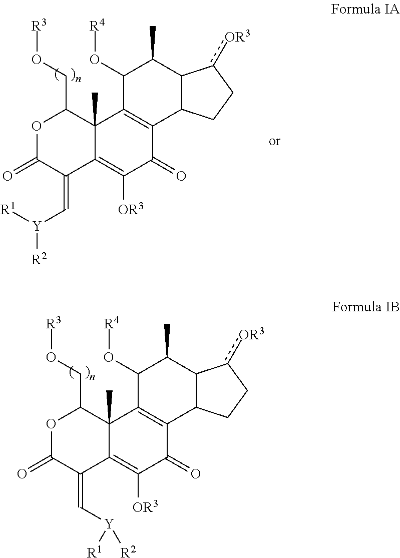

wherein:
- is an optional bond;
n is 1-6;
Y is a heteroatom
R¹ and R² are independently selected from an unsaturated alkyl, non-linear alkyl, cyclic alkyl, and substituted alkyl or R¹ and R² together with the atom to which they are attached form a heterocycloalkyl group;
R³ is absent, H, or C₁-C₆ substituted or unsubstituted alkyl;
R⁴ is (C═O)R⁵, (C═O)OR⁵, (S═O)R⁵, (SO₂)R⁵, (PO₃)R⁵, (C═O)NR⁵R⁶;
R⁵ is substituted or unsubstituted C₁-C₆ alkyl; and
R⁶ is substituted or unsubstituted C₁-C₆ alkyl.

In some embodiments, the compound of Formula IA or Formula IB is selected from:

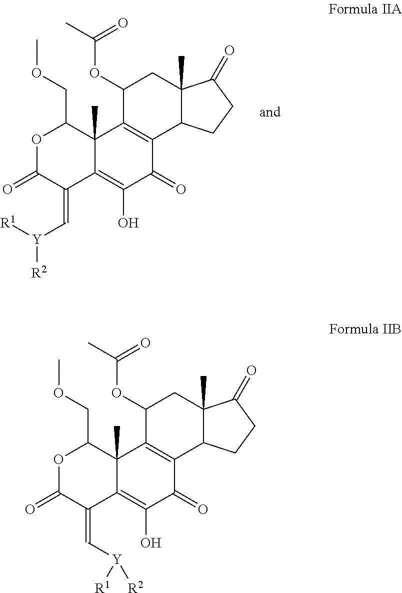

wherein Y is a heteroatom and R¹ and R² are independently selected from an unsaturated alkyl, non-linear alkyl, cyclic alkyl, and substituted alkyl.

In some embodiments, Y is a heteroatom selected from nitrogen and sulfur. In some embodiments, R¹ and R² are unsaturated alkyl. In some embodiments, the wortmannin analog is a PI-3 kinase inhibitor. In some embodiments, the PI-3 kinase inhibitor is PX-866. In some embodiments, the PI-3 kinase inhibitor is PX-867.

In some embodiments, the fibrosing syndrome is mild, moderate or severe pulmonary fibrosis, cystic fibrosis, ocular fibrosis (e.g., scarring post glaucoma filtration surgery), endomyocardial fibrosis, mediastinal fibrosis, myelofibrosis, osteofibrosis, fibrosing colonoapathy, retroperitoneal fibrosis, interstitial pneumonia, progressive massive fibrosis in lungs, keloids, scleroderma, hypertrophic scarring, renal fibrosis, intestinal fibrosis, liver fibrosis, fibrosing cholestatic hepatitis, nephrogenic systemic fibrosis, fibrosis associated with organ transplantation, multifocal fibrosclerosis, or anaphylactic shock fibrosis.

In some embodiments, the fibrosing syndrome is mild, moderate or severe idiopathic pulmonary fibrosis. In some embodiments, the fibrosing syndrome is pulmonary fibrosis associated with asbestosis, cystic fibrosis, infection, exposure to environmental allergens, lung transplant, autoimmune disease, or the fibrosing syndrome is drug-induced pulmonary fibrosis. In some embodiments, the fibrosing syndrome is associated with organ transplant.

Other objects, features and advantages of the methods, compounds, and compositions described herein will become apparent from the following detailed description. It should be understood, however, that the detailed description and the specific examples, while indicating specific embodiments, are given by way of illustration only. All references cited herein, including patents, patent applications, and publications, are hereby incorporated by reference for the purposes cited.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which principles of the invention are utilized, and the accompanying drawings of which:

FIG. 1 FIGS. 1A and 1B illustrates formulas for exemplary wortmannin analog and metabolite structures in accord with the present disclosure.

FIG. 2 PX-866 inhibits TGFα-induced phosphorylation of Akt (P-Akt). Western blot analysis of P-Akt levels in CCSP-rtTA/otet-TGFα transgenic mice increased over 5-fold after 1 day of Dox-induced TGFα expression compared to Dox-treated single transgene (CCSP/-) controls. Pretreatment of CCSP-rtTA/otet-TGFα mice with PX-866 prevented increased phosphorylation of Akt as demonstrated by representative immunoblotting (A) and densitometry analysis (B). Values are mean±SE, n=6 in each group. *P<0.05 compared to CCSP/-controls and PX-866-treated mice.

FIG. 3 PX-866 prevents establishment of pulmonary fibrosis. Sections of lungs from controls and CCSP-rtTA/otet-TGFα transgenic mice following 4 weeks of Dox were stained with trichrome (A). CCSP-rtTA/otet-TGFα transgenic mice administered PX-866 at the initiation of TGFα induction demonstrated marked attenuation of fibrosis compared to vehicle-treated CCSP-rtTA/otet-TGFα mice. Photomicrographs are from 2 separate animals and are representative of lungs from 5-7 mice in each group. All photomicrographs are taken at the same magnification and bar is 200 μm. Lung collagen content was determined from lungs of transgenic mice as described in Methods. PX-866 administered daily at the time of TGFα-induction prevented increases in lung collagen (B). Values are mean±SE. *p<0.05 compared to CCSP/-controls and PX-866-treated mice. +p<0.05 compared to CCSP-rtTA/otet-TGFα mice treated with vehicle.

FIG. 4 PX-866 prevents TGFα-dependent changes in lung function. Pulmonary mechanics were determined as described in Methods. PX-866 was administered daily at the time of TGFα-induction prevented increases in airway resistance, airway and tissue elastance, and decreases in compliance compared with vehicle-treated CCSP-rtTA/otet-TGFα transgenic mice receiving 4 weeks of Dox. *p<0.05 compared to CCSP/-controls and PX-866-treated mice. Data were derived from 6-10 mice in each group.

FIG. 5. PX-866 prevents progressive weight loss. To assess the efficacy of PI3K inhibition in established fibrosis, CCSP-rtTA/otet-TGFα transgenic mice were treated with PX-866 after 4 weeks of Dox while remaining on Dox for an additional 4 weeks. The treatment protocol is represented schematically in panel (A). Controls included CCSP/- and CCSP-rtTA/otet-TGFα mice treated with vehicle while remaining on Dox an additional 4 weeks. Mice were weighed weekly during treatments. Dox induced expression of TGFα for 8 weeks caused progressive weight loss in vehicle-treated mice (red line), while mice treated with PX-866 4 weeks after TGFα induction did not have changes in body weight (green line), but weights remained below CCSP/-controls (blue line), and CCSP-rtTA/otet-TGFα mice which received 4 weeks of Dox, then taken off Dox and treated with 4 weeks of vehicle (gold line) (B). *p<0.05 compared to CCSP/-control mice and CCSP-rtTA/otet-TGFα transgenic mice on and off Dox; # p<0.05 compared to PX-866-treated CCSP-rtTA/otet-TGFα transgenic mice. Data derived from 10 mice per group.

FIG. 6. PX-866 decreases progression of lung fibrosis. Both CCSP-rtTA/otet-TGFα transgenic mice administered PX-866 4 weeks after the initiation of TGFα induction and CCSP-rtTA/otet-TGFα transgenic mice taken off Dox demonstrated attenuation of fibrosis compared to vehicle-treated CCSP-rtTA/otet-TGFα mice (FIG. 6A). Photomicrographs are from 2 separate animals focused on pleural surface with adventitial (top) and alveolar areas (bottom). All photomicrographs are taken at the same magnification and are representative of lungs from 6 mice in each group. Lung collagen in PX-866-treated mice was unchanged compared with mice taken off Dox after 4 weeks (FIG. 6B), but remained elevated compared with CCSP/-controls. *p<0.05 compared to CCSP/-control mice and CCSP-rtTA/otet-TGFα transgenic mice on and off Dox; # p<0.05 compared to vehicle-treated CCSP-rtTA/otet-TGFα transgenic mice. Data derived from 10 mice per group.

FIG. 7. PX-866 slows progression of TGFα-dependent changes in lung mechanics. CCSP-rtTA/otet-TGFα transgenic mice administered PX-866 4 weeks after treatment with Dox demonstrated reduced increases in airway resistance, and airway and tissue elastance, and decreases in compliance compared with vehicle-treated CCSP-rtTA/otet-TGFα transgenic mice receiving 8 weeks of Dox. Lung mechanics in PX-866 mice were significantly altered compared with controls and CCSP-rtTA/otet-TGFα mice off Dox for 4 weeks. *p<0.05 compared to CCSP/-control mice and CCSP-rtTA/otet-TGFα transgenic mice on and off Dox; # p<0.05 compared to PX-866-treated CCSP-rtTA/otet-TGFα transgenic mice. Data derived from 10 mice per group.

### DETAILED DESCRIPTION OF THE INVENTION

Described herein are compounds, pharmaceutical compositions and medicaments that include PI-3 kinase inhibitors (e.g., compounds of Formula IA, IB, IIA, IIB or any other PI-3 kinase inhibitors described herein), and methods of using such compounds to treat or prevent diseases or conditions associated with PI-3 kinase activity. Also described herein, in some embodiments, are compounds that inhibit or partially inhibit PI-3 kinase activity (e.g., compounds of Formula IA, IB, IIA, IIB or any other PI-3 kinase inhibitors described herein), and methods of using such compounds and compositions for reversing or alleviating symptoms of fibrosing syndromes that are associated with PI-3 kinase activity. Also provided herein are wortmannin analogs and pharmaceutical compositions and medicaments that include wortmannin analogs for treatment of fibrosing syndromes.

In some instances, fibrosis is associated with proliferation of myofibroblasts and/or fibroblasts that express fibronectin. In some instances, survival of fibronectin-expressing myofibroblasts and/or fibroblasts in an affected organ is a determinant of progression of fibrosis. In some instances, fibronectin-mediated adhesion activates PI-3 kinase signaling pathways and contributes to onset and/or progression of fibrosis. In some instances, altered fibronectin expression and/or degradation in organ structure is associated with pathological manifestation of fibrosis.

In some instances, patients with lung cancer who are treated with EGFR tyrosine kinase inhibitors such as gefitinib or erlotinib develop drug-induced interstitial lung disease. In some embodiments, PI-3 kinase inhibitors described herein allow for treatment (e.g., reduction or reversal of fibrosis) of patients that develop drug-induced fibrosing syndromes such as interstitial lung disease. In some embodiments, methods of treatment described herein allow for treatment of fibrosing syndromes that are refractory to current methods of treatment (e.g., treatment with immune-suppressants, EFGR tyrosine kinase inhibitors).

The PI-3 kinases are a family of related enzymes that are capable of phosphorylating the 3 position hydroxyl group of the inositol ring of phosphatidylinositol. They are linked to a diverse list of cellular functions, including cell growth, proliferation, differentiation, motility, survival and intracellular trafficking Many of these functions relate to the ability of the PI-3 kinases to activate the protein kinase B (Akt). Genetic and pharmacological inactivation of the p110δ isoform of the PI-3 kinase has revealed this enzyme to be important for the function of T cells, B cell, mast cells and neutrophils. In some instances, PI-3 kinases play a role in the immune system response including initiation and/or maintenance of inflammatory responses. In some instances, inhibition of PI-3 kinase signaling inhibits extracellular matrix deposition, and reduces expression of profibrogenic factors. Profibrogenic factors include and are not limited to Connective tissue growth factor (CTGF), Fibroblast Growth Factor (FGF), Transforming Growth Factor alpha (TGF-α), Transforming Growth Factor beta (TGF-β), or the like.

In some instances PI3K-Akt is a primary downstream signaling pathway mediating EGFR-induced neoplastic processes, and mediates TGFα-induced fibrotic conditions (e.g., pulmonary fibrosis). In some instances inhibition of PI-3 kinase signaling in hepatic cells during active fibrogenesis inhibits extracellular matrix deposition and reduces expression of profibrogenic factors thereby reversing or reducing progression of hepatic fibrosis. See, Son et al. Hepatology. 2009, 50, 1512-23. In some instances, α8β1 is upregulated on myofibroblasts in fibrosis and other models of organ injury. In some instances, survival of α8 β1-expressing myofibroblasts is mediated by PI-3 kinase. In some instances, inhibition of PI-3 kinases reduces or reverses persistent fibrosis associated with organ injury. See, Farias et al. Biochemical and Biophysical Research Communications, 329, 2005, Pages 305-311.

Thus, inhibition of PI-3 kinase activity (e.g., via administration of compounds of Formula IA, IB, IIA, IIB or any other PI-3 kinase inhibitors described herein), reverses, reduces or delays progression of fibrosis in an individual in need thereof. In addition, PI-3 kinase inhibition (e.g., via administration of compounds of Formula IA, IB, IIA, IIB or any other PI-3 kinase inhibitors and/or wortmannin analogs described herein), alleviates and/or treats established fibrosis after fibrosis is pronounced and progressing. In some embodiments, inhibition of PI-3 kinase activity (e.g., via administration of compounds of Formula IA, IB, IIA, IIB or any other PI-3 kinase inhibitors and/or wortmannin analogs described herein), delays onset of fibrosis in individuals pre-disposed to a fibrosing syndrome (e.g., an individual with a family history of cystic fibrosis). In some embodiments, inhibition of PI-3 kinase activity (e.g., via administration of compounds of Formula IA, IB, IIA, IIB or any other PI-3 kinase inhibitors and/or wortmannin analogs described herein), reduces or prevents the occurrence of fibrosis (e.g., after organ transplantation).

Accordingly, described herein are methods of reducing or partially reducing activity of PI-3 kinases in individuals in need thereof, thereby reversing fibrosis or delaying the progression of fibrosis. In some embodiments, the methods comprise administration of PI-3 kinase inhibitors (e.g., compounds of Formula IA, IB, IIA, IIB or any other PI-3 kinase inhibitors and/or wortmannin analogs described herein), to individuals in need thereof. In some embodiments, PI-3 kinase inhibitors described herein are reversible PI-3 kinase inhibitors. In other embodiments, PI-3 kinase inhibitors described herein are irreversible PI-3 kinase inhibitors. In some embodiments, PI-3 kinase inhibitors described herein are more potent inhibitors of PI-3 kinase alpha or PI-3 kinase beta compared to inhibitory activity towards PI-3 kinase delta or PI-3 kinase gamma.

### Certain Definitions

It must also be noted that as used herein and in the appended claims, the singular forms “a”, “an”, and “the” include plural reference unless the context clearly dictates otherwise. Thus, for example, reference to a “cell” is a reference to one or more cells and equivalents thereof known to those skilled in the art, and so forth. Unless defined otherwise, all technical and scientific terms used herein have the same meanings as commonly understood by one of ordinary skill in the art. Although any methods and materials similar or equivalent to those described herein can be used in the practice or testing of embodiments described herein, certain preferred methods, devices, and materials are now described.

As used herein, the term “about” means plus or minus 10% of the numerical value of the number with which it is being used. Therefore, about 50% means in the range of 45%-55%. “Optional” or “optionally” may be taken to mean that the subsequently described structure, event or circumstance may or may not occur, and that the description includes instances where the events occurs and instances where it does not.

“Administering” when used in conjunction with a therapeutic means to administer a therapeutic systemically or locally, as directly into or onto a target tissue, or to administer a therapeutic to a patient whereby the therapeutic positively impacts the tissue to which it is targeted. Thus, as used herein, the term “administering”, when used in conjunction with a wortmannin analog or metabolite thereof, can include, but is not limited to, providing a wortmannin analog or metabolite thereof into or onto the target tissue; providing a wortmannin analog or metabolite thereof systemically to a patient by, e.g., intravenous injection whereby the therapeutic reaches the target tissue or cells. “Administering” a composition may be accomplished by injection, topical administration, and oral administration or by other methods alone or in combination with other known techniques.

As used herein, the term “therapeutic” means an agent utilized to treat, combat, ameliorate, prevent or improve an unwanted condition or disease of a patient. In some embodiments, a therapeutic agent is directed to the treatment and/or the amelioration of or reversal of the symptoms of a fibrotic condition described herein. In some embodiments, a therapeutic agent described herein is directed to treatment of pulmonary fibrosis and/or the amelioration of or reversal of the symptoms of pulmonary fibrosis.

The term “animal” as used herein includes, but is not limited to, humans and non-human vertebrates such as wild, domestic and farm animals. The terms “patient” and “subject” and “individual” are interchangeable and may be taken to mean any living organism which may be treated with compounds of the present disclosure. As such, the terms “patient” and “subject” may include, but are not limited to, any non-human mammal, any primate or a human.

The term “inhibiting” includes the administration of a compound of the present disclosure to prevent the onset of symptoms, alleviate symptoms, or eliminate the disease, condition or disorder.

By “pharmaceutically acceptable”, it is meant the carrier, diluent or excipient must be compatible with the other ingredients of the formulation and not deleterious to the recipient thereof.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

A “therapeutically effective amount” or “effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing the pathology and/or symptomatology). As such, a non-limiting example of a “therapeutically effective amount” or “effective amount” of a composition of the present disclosure may be used to inhibit, block, or reverse the activation, migration, or proliferation of cells or to effectively treat cancer or ameliorate the symptoms of cancer.

The terms “fibrosis” or “fibrosing syndrome” or “fibrotic condition” are used interchangeably. As used herein, the terms “fibrosis” or “fibrosing syndrome” or “fibrotic condition” refer to conditions that follow acute or chronic inflammation and/or injury and are associated with the abnormal accumulation of cells and/or collagen at the site of inflammation or injury and include, but are not limited to, fibrosis of individual organs or tissues such as the heart, kidney, joints, lung, or skin. The terms “fibrosis” or “fibrosing syndrome” or “fibrotic condition” include interstitial lung disease including pulmonary fibrosis, idiopathic pulmonary fibrosis, cryptogenic fibrosing alveolitis, ocular fibrosis (e.g., scarring associated with age related macular degeneration, or scarring after glaucoma filtration surgery), cystic fibrosis, endomyocardial fibrosis, mediastinal fibrosis, myelofibrosis, osteofibrosis, fibrosing colonoapathy, retroperitoneal fibrosis, interstitial pneumonia, progressive massive fibrosis in lungs (a complication of coal workers' pneumoconiosis), keloids, scleroderma, hypertrophic scarring, renal fibrosis (e.g., tubulointerstitial fibrosis), intestinal fibrosis (e.g., associated with Crohn's disease, Inflammatory Bowel disease), liver fibrosis, fibrosing cholestatic hepatitis, nephrogenic systemic fibrosis, multifocal fibrosclerosis, anaphylactic shock fibrosis, or the like. In some embodiments, any fibrosis or fibrosing syndrome described herein is of unknown origin (idiopathic). In some embodiments, any fibrosis or fibrosing syndrome described herein is associated with cystic fibrosis. In some embodiments, any fibrosing syndrome described herein is associated with autoimmune disease, inflammation, cancer, or the like. In some embodiments, any fibrosing syndrome described herein is associated with infection (e.g., pneumonia, tuberculosis, avian flu or the like). In some embodiments, any fibrosis or fibrosing syndrome described herein is associated with organ transplant (e.g., lung transplant, liver transplant, kidney transplant). In some embodiments, any fibrosis or fibrosing syndrome described herein is associated with exposure to allergens and/or environmental pollutants including and not limited to asbestos, coal dust, cigarette smoke, diesel exhaust, ozone, atmospheric particulates or the like. In some embodiments, any fibrosis or fibrosing syndrome described herein is drug-induced fibrosis.

The present methods include both medical therapeutic and/or prophylactic treatment, as appropriate. The specific dose of a compound administered to obtain therapeutic and/or prophylactic effects will, of course, be determined by the particular circumstances surrounding the case, including, for example, the compound administered, the route of administration, and the condition being treated. The compounds are effective over a wide dosage range and, for example, dosages per day will normally fall within the range of from 0.001 to 100 mg/kg, more usually in the range of from 0.01 to 1 mg/kg. However, it will be understood that the effective amount administered will be determined by the physician in light of the relevant circumstances including the condition to be treated, the choice of compound to be administered, and the chosen route of administration. A therapeutically effective amount of compound described herein is typically an amount such that when it is administered in a physiologically tolerable excipient composition, it is sufficient to achieve an effective systemic concentration or local concentration in the tissue.

The terms “treat,” “treated,” or “treating” as used herein refers to both therapeutic treatment and prophylactic or preventative measures, wherein the object is to prevent or slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

The term “wortmannin analog” or “analog of wortmannin” refers to any compounds in which one or more atoms, functional groups, or substructures in wortmannin have been replaced with different atoms, groups, or substructures while retaining or improving upon the functional activity of wortmannin and/or improving PK profiles and/or reducing toxicity of wortmannin.

An “alkyl” group refers to an aliphatic hydrocarbon group. An “alkyl” group includes substituted and unsubstituted alkyl groups. Reference to an alkyl group includes “saturated alkyl” and/or “unsaturated alkyl”. The alkyl group, whether saturated or unsaturated, includes branched, straight chain, or cyclic groups. By way of example only, alkyl includes methyl, ethyl, propyl, iso-propyl, n-butyl, iso-butyl, sec-butyl, t-butyl, pentyl, iso-pentyl, neo-pentyl, and hexyl. In some embodiments, alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. A “lower alkyl” is a C₁-C₆ alkyl. A “heteroalkyl” group substitutes any one of the carbons of the alkyl group with a heteroatom having the appropriate number of hydrogen atoms attached (e.g., a CH₂ group to an NH group or an 0 group).

The term “cycloalkyl” or “cyclic alkyl” refers to a monocyclic or polycyclic non-aromatic radical, wherein each of the atoms forming the ring (i.e. skeletal atoms) is a carbon atom. A “cycloalkyl” group includes substituted and unsubstituted cycloalkyl groups. In various embodiments, cycloalkyls are saturated, or partially unsaturated. In some embodiments, cycloalkyls are fused with an aromatic ring. In some embodiments, cycloalkyls are fused with a heteroaryl ring. Cycloalkyl groups include groups having from 3 to 10 ring atoms. Illustrative examples of cycloalkyl groups include, but are not limited to, the following moieties:

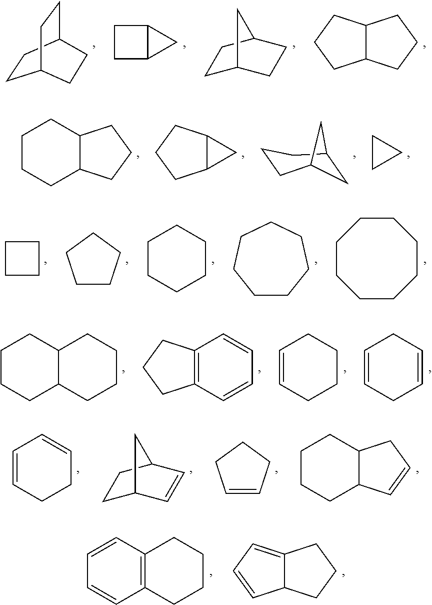

and the like. Monocyclic cycloalkyls include, but are not limited to, cyclopropyl, cyclobutyl, cyclopentyl, cyclohexyl, cycloheptyl, and cyclooctyl. Dicylclic cycloalkyls include, but are not limited to tetrahydronaphthyl, indanyl, tetrahydropentalene or the like. Polycyclic cycloalkyls include admantane, norbornane or the like. The term cycloalkyl includes “unsaturated nonaromatic carbocyclyl” or “nonaromatic unsaturated carbocyclyl” both of which refer to a nonaromatic carbocyclyle, as defined herein, that contains at least one carbon carbon double bond or one carbon carbon triple bond.

A “heteroalicyclic” group or “heterocyclo” group or “heterocycloalkyl” group refers to a cycloalkyl group, wherein at least one skeletal ring atom is a heteroatom selected from nitrogen, oxygen and sulfur. A “heterocycloalkyl” group includes substituted and unsubstituted heterocycloalkyl groups. In various embodiments, the radicals are fused with an aryl or heteroaryl. Illustrative examples of heterocyclo groups, also referred to as non-aromatic heterocycles, include:

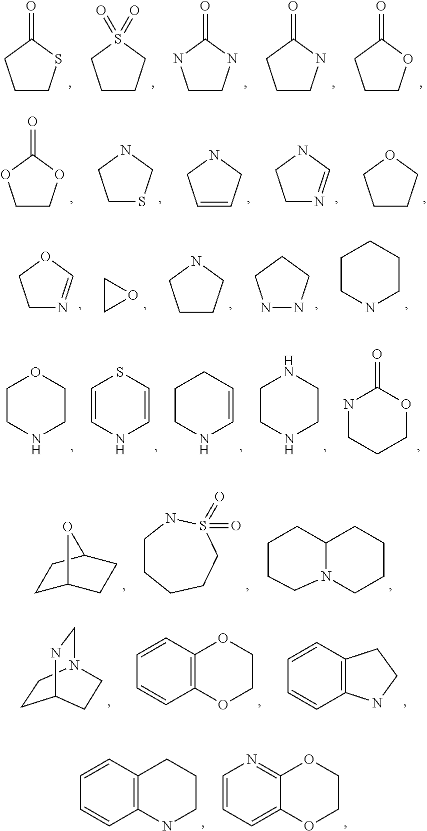

and the like. The term heteroalicyclic also includes all ring forms of the carbohydrates, including but not limited to the monosaccharides, the disaccharides and the oligosaccharides.

### Wortmannin Analogs

Wortmannin is a naturally occurring compound isolated from culture broths of the fungus Penicillium wortmannin. Wortmannin irreversibly inhibits PI-3-kinase through covalent interaction with a specific lysine on the kinase: Lys⁸⁰² of the ATP binding pocket of the catalytic site of the pi 10a isoform or Lys⁸⁸³ of the pi 105 isoform. Most isoforms of PI-3 kinase, such as p110α, p110β, p110δ and p110γ for example, are inhibited equally by wortmannin. Wortmannin demonstrates liver and hematologic toxicity, however, and is a biologically unstable molecule. Samples stored as aqueous solutions at either 37° C. or 0° C. at neutral pH are subject to decomposition by hydrolytic opening of the furan ring. It has been shown that the electrophilicity of the furan ring is central to the inhibitory activity of wortmannin. The irreversible inhibition of PI-3-kinase occurs by formation of an enamine following the attack of the active lysine of the kinase on the furan ring at position C(20) of wortmannin. Thus, decomposition of wortmannin interferes with its inhibitory activity on PI-3 kinases.

In some embodiments, analogs and metabolites of wortmannin described herein display improved biological stability and reduced systemic toxicity. In some embodiments, analogs and metabolites of wortmannin described herein are PI-3 kinase inhibitors. Accordingly, methods and compositions of wortmannin analogs described herein allow for improved methods of treating fibrotic conditions including, for example, pulmonary fibrosis.

In some embodiments, wortmannin analogs suitable for methods of treatment described herein include compounds of Formula IA or IB:

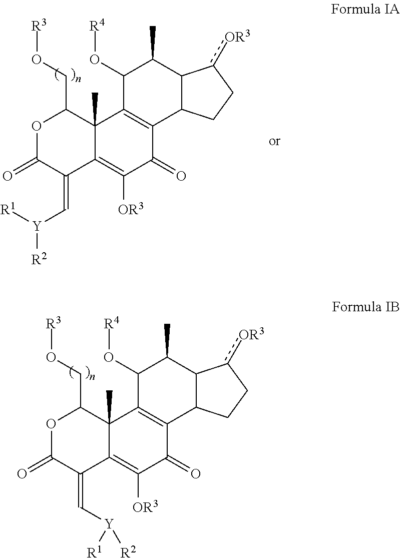

wherein:
- is an optional bond;
n is 1-6;
Y is a heteroatom
R¹ and R² are independently selected from an unsaturated alkyl, non-linear alkyl, cyclic alkyl, and substituted alkyl or R¹ and R² together with the atom to which they are attached form a heterocycloalkyl group;
R³ is absent, H, or C₁-C₆ substituted or unsubstituted alkyl;
R⁴ is (C═O)R⁵, (C═O)OR⁵, (S═O)R⁵, (SO₂)R⁵, (PO₃)R⁵, (C═O)NR⁵R⁶;
R⁵ is substituted or unsubstituted C₁-C₆ alkyl; and
R⁶ is substituted or unsubstituted C₁-C₆ alkyl.

In some embodiments, wortmannin analogs suitable for methods of treatment described herein include compounds of formula:

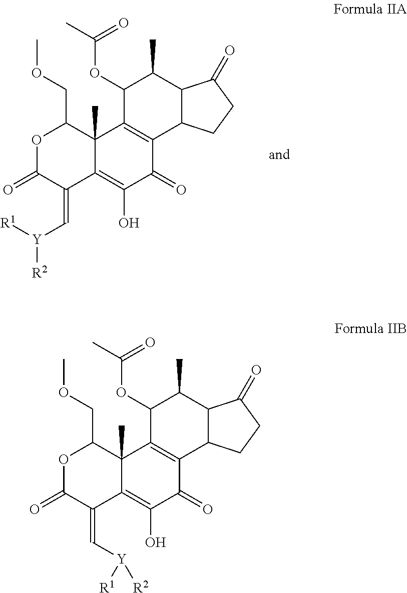

wherein Y is a heteroatom and R¹ and R² are independently selected from an unsaturated alkyl, non-linear alkyl, cyclic alkyl, and substituted alkyl.

In some embodiments, wortmannin analogs suitable for treatment of fibrosing syndromes described herein include compounds and/or metabolites thereof selected from, but not limited to, PX-866, PX-867, PX-868, PX-870, PX-871, PX-880, PX-881, PX-882, PX-889, PX-890, DJM2-170, DJM2-171, DJM2-177, DJM2-181 and combinations thereof. In some embodiments, wortmannin analogs suitable for treatment of fibrosing disorders described herein include compounds described in GB2302021 which compounds are incorporated herein by reference.

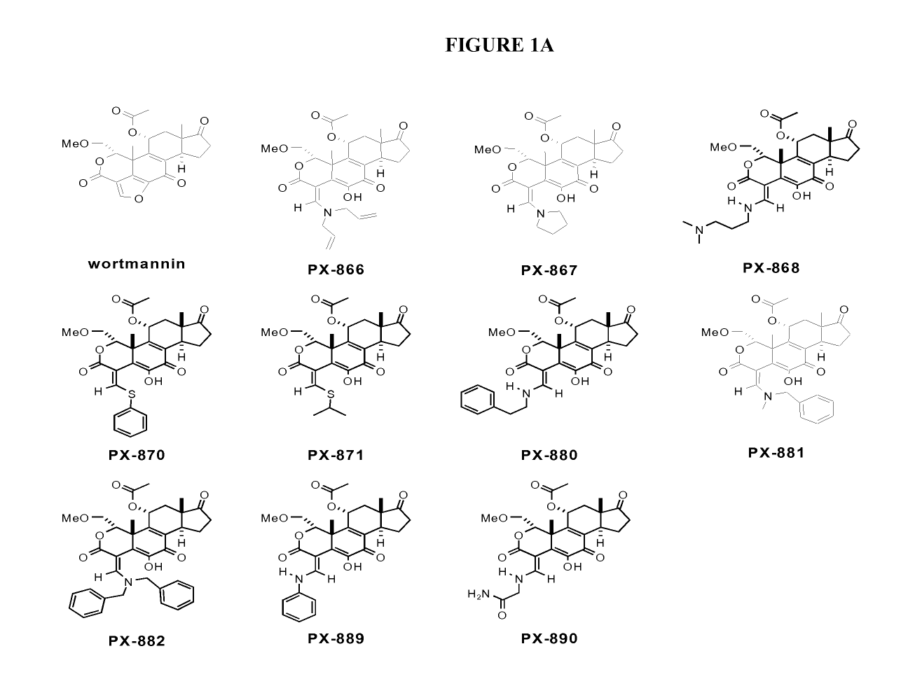

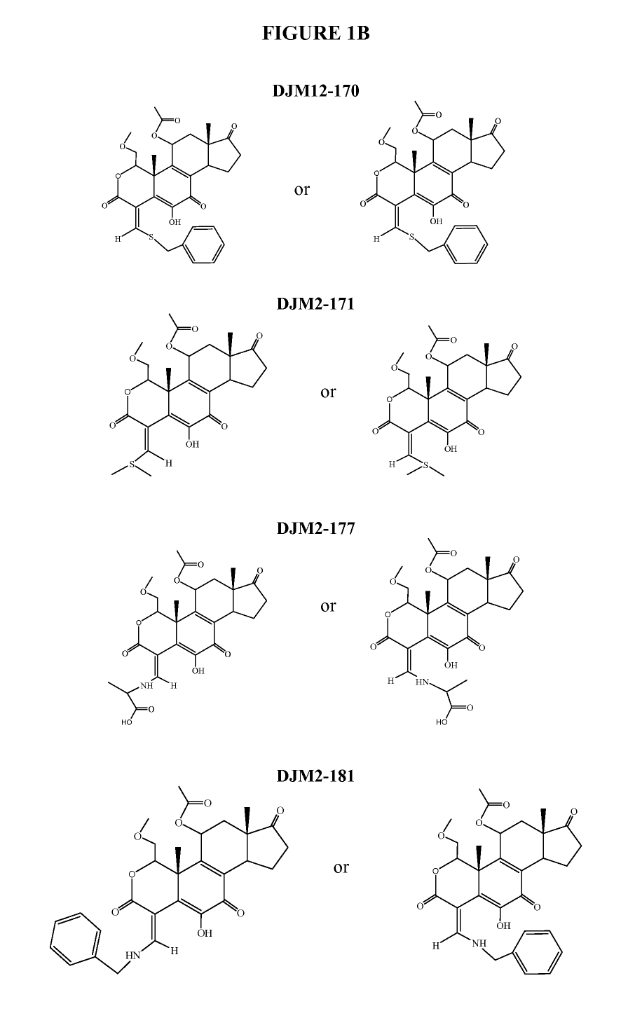

FIG. 1 illustrates formulas for exemplary wortmannin analogs and metabolites thereof that are useful in treatment of fibrosing syndromes.

### PI-3 Kinase Inhibitors

In some embodiments, PI-3 kinase inhibitors suitable for treatment of fibrosing syndromes (e.g., pulmonary fibrosis) described herein include, but are not limited to, wortmannin analogs, wortmannin metabolites, NVP-BEZ235, PI-130, LY294002 and all-trans-retinoic-acid (ATRA).

In some embodiments, PI-3 kinase inhibitors suitable for treatment of fibrosing syndromes described herein include compounds of Formula IA, IB, IIA and IIB as described herein.

In some embodiments, PI-3 kinase inhibitors suitable for treatment of fibrosing syndromes (e.g., pulmonary fibrosis) include and are not limited to PI-3 kinase inhibitors described in U.S. Appl. Publication Nos. 20050032727, 20070203098, 20070259876, 20080188423, 20090042773, 20070021447, 20080039459, 20080300239, 20090018131, 20090023742, 20090029998, 20090048252, 20090170848, 20090215818, 20090306074, 20090048252, 20080300239, 20090018131, 20090023742, 20090048252, 20090170848, 20090215818, 20090306074, PI-3 kinase inhibitor compounds described therein are hereby incorporated herein by reference.

### Fibrosing Syndromes and Methods of Treatment

### Pulmonary Fibrosis

Provided herein are methods of treating interstitial lung disease comprising administration of one or more inhibitors of PI-3 kinases to an individual in need thereof. In some embodiments, the interstitial lung disease is pulmonary fibrosis. In some embodiments, the interstitial lung disease is idiopathic pulmonary fibrosis.

Pulmonary fibrosis contributes to morbidity and mortality in a number of pediatric and adult lung diseases. Clinical diseases causing pulmonary fibrosis are heterogeneous and fibrosis may develop secondary to acute lung injury such as in acute respiratory distress syndrome, from chronic inflammatory diseases such as in cystic fibrosis (CF), or may develop of unknown cause as in idiopathic pulmonary fibrosis (IPF). While the pathologic features of pulmonary fibrosis may vary depending on the underlying disease process, a number of common characteristics are present including mesenchymal cell proliferation, expansion of the extracellular matrix and remodeling of the lung parenchyma. As used herein, pulmonary fibrosis includes idiopathic pulmonary fibrosis, diffuse interstitial pulmonary fibrosis, interstitial pneumonitis, progressive massive fibrosis in lungs (a complication of coal workers' pneumoconiosis), or the like. Also contemplated within the scope of embodiments described herein is pulmonary fibrosis that arises from underlying disease such as cystic fibrosis, or autoimmune disease such as scleroderma or the like. As used herein, pulmonary fibrosis includes pulmonary fibrosis arising from exposure to environmental allergens or pollutants including, but not limited to asbestos (i.e., pulmonary fibrosis associated with asbestosis), coal dust, cigarette smoke, diesel exhaust, other atmospheric pollutants such as ozone, particulates from industry emissions or the like. As used herein, pulmonary fibrosis includes pulmonary fibrosis associated with infection such as pneumonia or any other infections. Pulmonary fibrosis also includes drug-induced pulmonary fibrosis (e.g., fibrosis arising as a side-effect from administration of drugs such as bleomycin or the like).

Accordingly, the methods and compositions provided herein reduce, reverse, or delay progression and/or onset of any interstitial lung disorder and/or pulmonary fibrosing syndrome described herein. In some embodiments, interstitial lung disease and/or a pulmonary fibrosing syndrome is associated with proliferation of myofibroblasts in lungs. In some embodiments, administration of one or more PI-3 kinase inhibitors (e.g., compounds of Formula IA, IAB, IIA or IIB or any other PI-3 kinase inhibitor described herein) to an individual in need thereof inhibits or partially inhibits proliferation of myofibroblasts in lungs, thereby reducing, reversing, or delaying progression and/or onset of any interstitial lung disease and/or pulmonary fibrosing syndrome described herein.

EGFR (HER1) belongs to a receptor tyrosine kinases protein family which also includes HER2/neu, HER3 and HER4. Six EGFR ligands (TGFα, EGF, HB-EGF, amphiregulin, betacellulin, and heregulin) have been localized to the lung or in lung cells. Depending on the activating ligand, EGFR family members form various homo- or heterodimers with different biological capacities. Activation of the EGFR regulates diverse cellular functions, many of which are associated with fibrogenesis, including cell growth, proliferation, differentiation, migration, protection from apoptosis, and transformation. Doxycycline (Dox) regulatable transgenic mice that specifically express TGFα in the lung epithelium, show progressive and extensive vascular adventitial, peribronchial, interstitial and pleural fibrosis that is independent of inflammation. Gene expression profiles observed after expression of TGFα in these mice lungs are similar to those found in pulmonary fibrotic disease in humans.

In some instances, signaling pathways downstream of EGFR activation mediate TGFα-induced pulmonary fibrosis. Following ligand binding to the extracellular domain, receptor homo- and heterodimers are formed leading to auto- or trans-phosphorylation by the intrinsic tyrosine-kinase activity on specific residues in the cytoplasmic domains. The phosphorylated tyrosine residues become docking sites for signaling molecules that activate multiple downstream effector pathways including the RAS/RAF/mitogen-activated protein kinase (MAPK) cascade, the JAK/STAT pathway, the phospholipase Cγ pathway and the phosphatidylinositol 3′-kinase (PI3K)/Akt (protein Kinase B) signaling pathway. PI3K is a signal transduction enzyme that catalyzes the phosphorylation of phosphatidylinositol (4,5)-biphosphate (PIP2) to form phosphatidylinositol (3,4,5)-triphosphate (PIP3) in response to activation of receptor tyrosine kinases, G-protein coupled receptors or cytokine receptors. PIP3 in turn activates Akt and has been associated with a number of cellular processes associated with fibrogenesis including growth, proliferation, migration, survival and collagen gene expression. Tumor suppressor phosphatase and tensin homolog (PTEN) is a negative growth regulator of the PI3K-Akt pathway that dephosphorylates PIP3 to PIP2. Both PTEN haploinsufficient mice and wild type mice treated with a pharmacologic inhibitor of PTEN demonstrate augmented collagen deposition and myofibroblast differentiation following bleomycin-induced lung injury supporting a role in vivo for unopposed PI3K-Akt activation in the pathogenesis of pulmonary fibrosis.

In some embodiments, administration of one or more PI-3 kinase inhibitors to an individual in need thereof reduces or suppresses activation of EGFR in lung cells. In some embodiments, administration of one or more PI-3 kinase inhibitors to an individual in need thereof reduces or hinders binding of TGF-α to EGFR thereby inhibiting or partially inhibiting the downstream activation of PI-3 kinases.

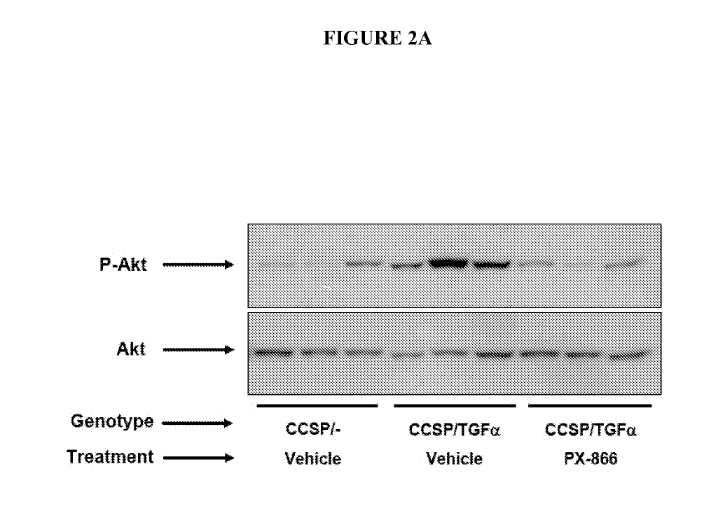

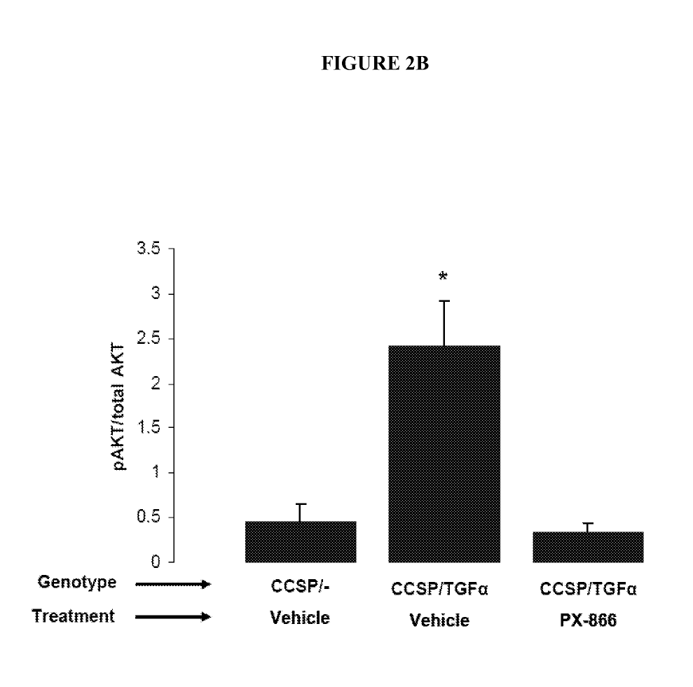

Since PBK-Akt is a primary downstream signaling pathway mediating EGFR-induced neoplastic processes, PBK-Akt may mediate TGFα-induced pulmonary fibrosis. PX-866 is a novel inhibitor of PI3K that is currently in advanced preclinical development as an antitumor agent. The role of PBK in the initiation and propagation of pulmonary fibrosis by administering PX-866 at the time of TGFα induction in regulatable transgenic mice is described herein in FIGS. 2-7 and Examples 1-2.

In some instances, additional downstream signaling pathways other than PI3K remain activated and continue to contribute towards maintenance of lung fibrosis. Tyrosine kinase receptors such as EGFR activate the PI3K and other pathways which control cellular growth and proliferation. In some embodiments, targeted therapeutic intervention in additional signaling pathways active in the maintenance of lung fibrosis allows for combination therapy of lung fibrosis.

Examples 1-2 and FIGS. 2-7 demonstrate that treatment with the PI3K inhibitor PX-866 prevents EGFR-mediated pulmonary fibrosis and associated alterations in lung mechanics in transgenic mice. Increased EGFR ligands and activation of EGFR have been identified in several studies of patients with fibrotic lung disease. Increased TGFα was detected in the lung lavage fluid of patients with IPF, and immunohistochemistry localized increases in TGFα and EGFR to type II epithelial cells, fibroblasts and the vascular endothelium of IPF samples. Increased EGFR and EGFR ligands have also been identified in remodeled tissue of patients with cystic fibrosis, bronchopulmonary dysplasia and asthma. In some instances, EGFR-targeted therapy blocks fibrosis in a number of animal models including bleomycin, naphthalene, asbestosis and ovalbumin models of lung fibrosis. Accordingly, also contemplated within the scope of embodiments described herein is combination therapy comprising administration of EGFR-targeted therapeutics and PI-3 kinase modulators for treatment of lung fibrosis.

In some instances, EGFR signaling mediates interstitial lung disease and maintains surfactant protein expression during acute lung injury. In certain instances inhibition of EGFR exacerbates lung injury by reducing surfactant protein expression. Together, these findings support further analysis of signaling pathways downstream of EGFR that mediate fibrosis with the goal of defining pathways which are specific to lung remodeling. This disclosure demonstrates that PI3K-Akt is the primary effector pathway downstream of EGFR activation mediating pulmonary fibrosis.

PI3K pathway is involved in mediating lung fibrosis. Studies in both human and mouse fibroblasts demonstrate that PI3K activation leads to reduced apoptosis along with increased proliferation, collagen synthesis and myofibroblasts differentiation. Fibroblasts isolated from patients with IPF demonstrate decreased PTEN expression and activity associated with aberrant activation of PI3K-Akt and increased proliferation. Pulmonary fibrosis in the regulatable TGFβ1 transgenic model was significantly attenuated when mice were treated with an Akt inhibitor.

Fibrosis in the TGFα transgenic model develops independent of TGFα activation suggesting the PI3K/Akt pathway may represent a potential point of confluence where multiple pro-fibrotic stimuli converge to elicit the cellular response of mesenchymal proliferation and matrix deposition. The platelet derived growth factor (PDGF) family is another profibrotic cytokine family implicated in inflammatory models of lung fibrosis. PDGFs act via two receptors which, like EGFR, are receptor tyrosine kinases. Like EGFR and TGFβ1, PDGF receptors activate PI3K. Collectively, these data further support the PI3K as a common pathway where multiple fibrogenic cytokines converge.

Both epithelial cells and mesenchymal cells proliferate in response to TGFα, however it is unclear if PI3K activation in both cell types leads to fibrosis. Transgenic mice in which the Pten gene was conditionally deleted from the pulmonary epithelium demonstrated increased epithelial PI3K-Akt activation associated with marked epithelial hyperplasia characterized by a hypercellular epithelium lining papillae with fibrovascular cores that protruded into bronchial and bronchiolar lumens. However, unlike TGFα mice, the hyperplasia was not progressive and parenchymal fibrosis did not develop suggesting PI3K activation of the fibroblast is important in mediating PI3K/Akt-mediated fibrosis.

Recent data support activation of PI3K-Akt in human fibrotic lung disease. Immunohistochemical analysis of lung biopsies from IPF patients demonstrate increased phosphorylated Akt in fibroblastic foci. PI3K inhibition in TGFα and TGFβ transgenic models coupled with evidence of aberrant PI3K signaling in human fibrotic disease supports pharmacologically targeting the PI3K-Akt pathway.

In some embodiments, provided herein are methods of treating pulmonary fibrosis in a subject comprising administering to a subject a therapeutically effective amount of a wortmannin analog or a wortmannin metabolite.

In some embodiments, provided herein are methods for the use of PI-3 kinase inhibitors (e.g., compounds of Formula IA, IB, IIA or IIB, or any other PI-3 kinase inhibitor described herein) for treatment of pulmonary fibrosis, wherein the pulmonary fibrosis is mild or moderate. In some embodiments, provided herein are methods for the use of PI-3 kinase inhibitors (e.g., compounds of Formula IA, IB, IIA or IIB, or any other PI-3 kinase inhibitor described herein) for treatment of pulmonary fibrosis, wherein the pulmonary fibrosis is pronounced and progressing. In some embodiments, a PI-3 kinase inhibitor (e.g., compounds of Formula IA, IB, IIA or IIB, or any other PI-3 kinase inhibitor described herein) decreases progression of lung fibrosis. In some embodiments, a PI-3 kinase inhibitor (e.g., compounds of Formula IA, IB, IIA or IIB, or any other PI-3 kinase inhibitor described herein) slows progression of TGF-α-dependent changes in lung mechanics. In some embodiments, a PI-3 kinase inhibitor (e.g., compounds of Formula IA, IB, IIA or IIB, or any other PI-3 kinase inhibitor described herein) prevents progressive weight loss.

In some embodiments, provided herein are methods of treating pulmonary fibrosis comprising administration of metabolites of compounds of Formula IA, IB, IIA or IIB. By way of example, certain metabolites are shown in FIG. 1. In some of such embodiments, such metabolites demonstrate inhibitory activities against PI-3 kinases that are similar to or better than inhibitor activity of wortmannin.

### Ocular Fibrosis

Provided herein are methods of treatment of ocular fibrosis (fibrosis and/or scarring in any region of an eye) comprising administration of one or more wortmannin analogs and/or inhibitors of PI-3 kinases (e.g., compounds of Formula IA, IB, IIA or IIB) to an individual in need thereof. In some instances, when ocular homeostasis is disturbed, e.g., by infection or inflammation or metabolic disease, fibrosis is mediated by glial cells and/or fibroblasts. In some instances, fibrosis of cornea (e.g., herpetic keratitis) occurs after an infection (e.g. a viral infection). In some instances, diabetes-associated retinal hypoxia leads to fibrosis and subsequent traction retinal detachment (a complication of advanced diabetic retinopathy). In some instances, subretinal hemorrhaging associated with neovascular age-related macular degeneration (ARMD) causes fibrosis under the retina. In some instances, proliferation of fibroblasts and fibroblast-like cells (e.g., glial cells in the eye) leads to modification of extracellular matrix, leading to scar formation and/or loss of vision. In some instances, degeneration of conjunctiva results in fibrosis at the corneal surface. In some instances, ocular fibrosis occurs subsequent to a corneal transplant. In some instances, retinopathy of prematurity (ROP) is associated with fibrosis in eyes of premature infants. In some instances, scarring occurs post glaucoma filtration surgery.

In some instances, ocular fibrosis is a result of a shift in the balance between levels of pro-angiogenic VEGF and pro-fibrotic CTGF. In some embodiments, administration of one or more PI-3 kinase inhibitors to an individual in need thereof inhibits or partially inhibits CTGF. In some embodiments, inhibition or partial inhibition of CTGF reduces, reverses, or delays progression and/or onset of ocular fibrosis. Accordingly, the methods and compositions provided herein reduce, reverse, or delay progression and/or onset of any ocular fibrosing syndrome described herein.

In some embodiments, an ocular fibrosing syndrome is associated with proliferation of fibroblasts or fibroblast-like cells. In some embodiments, administration of one or more wortmannin analogs and/or PI-3 kinase inhibitors (e.g., compounds of Formula IA, IB, IIA or IIB) to an individual in need thereof inhibits or partially inhibits proliferation of fibroblasts, thereby reducing, reversing, or delaying progression and/or onset of any ocular fibrosing syndrome described herein. In some embodiments, administration of one or more wortmannin analogs and/or PI-3 kinase inhibitors (e.g., compounds of Formula IA, IB, IIA or IIB) to an individual in need thereof reduces, reverses, or delays progression and/or onset of scarring post-glaucoma filtration surgery. In some embodiments, administration of one or more wortmannin analogs and/or PI-3 kinase inhibitors (e.g., compounds of Formula IA, IB, IIA or IIB) to an individual in need thereof reduces, reverses, or delays progression and/or onset of scarring in the retina associated with ARMD.

### Fibrosing Syndromes in the Gastrointestinal Tract

Provided herein are methods of treatment of fibrosing syndromes in the gastrointestinal (GI) tract comprising administration of one or more wortmannin analogs and/or inhibitors of PI-3 kinases (e.g., compounds of Formula IA, IB, IIA or IIB) to an individual in need thereof. Fibrosing syndromes in the GI tract include and are not limited to fibrosing colonoapathy, intestinal fibrosis (e.g., associated with Crohn's disease, Inflammatory Bowel disease), liver fibrosis, fibrosing cholestatic hepatitis, or the like. In some of such embodiments, a GI tract fibrosing syndrome is associated with cystic fibrosis (e.g., fibrosing colonaphthy). In some instances, activated fibroblasts contribute to fibrotic extracellular matrix accumulation during liver fibrosis.

In some embodiments, a GI tract fibrosing syndrome is associated with proliferation of fibroblasts. In some embodiments, administration of one or more wortmannin analogs and/or PI-3 kinase inhibitors (e.g., compounds of Formula IA, IB, IIA or IIB) to an individual in need thereof inhibits or partially inhibits proliferation of fibroblasts, thereby reducing, reversing, or delaying progression and/or onset of any GI tract fibrosing syndrome described herein.

### Fibrosing Syndromes in the Renal System

Provided herein are methods of treatment of fibrosing syndromes in the renal system comprising administration of one or more wortmannin analogs and/or inhibitors of PI-3 kinases (e.g., compounds of Formula IA, IB, IIA or IIB) to an individual in need thereof. Fibrosing syndromes in the renal system include and are not limited to chronic kidney disease, retroperitoneal fibrosis, diabetic nephropathy, chronic glomerulosclerosis, tubulointerstitial fibrosis, or the like.

In some embodiments, a renal fibrosing syndrome is associated with proliferation and/or activation of fibroblasts. In some embodiments, administration of one or more wortmannin analogs and/or PI-3 kinase inhibitors (e.g., compounds of Formula IA, IB, IIA or IIB) to an individual in need thereof inhibits or partially inhibits proliferation of fibroblasts, thereby reducing, reversing, or delaying progression and/or onset of any renal fibrosing syndrome described herein.

### Dermal Fibrosing Syndromes

Provided herein are methods of treatment of dermal fibrosing syndromes comprising administration of one or more wortmannin analogs and/or inhibitors of PI-3 kinases (e.g., compounds of Formula IA, IB, IIA or IIB) to an individual in need thereof. Dermal fibrosing syndromes include and are not limited to keloids, scleroderma, hypetrophic scarring, nephrogenic systemic fibrosis, or the like. In some instances, keloids are the result of an overgrowth of dense fibrous tissue that develops after healing of a skin injury. In some instances, hypertrophic scars are visible after thermal injuries and/or other injuries that involve the deep dermis. Nephrogenic Systemic Fibrosis (NSF) is a systemic disorder with prominent and visible effects in the skin. In some instances, patients diagnosed with NSF develop large areas of hardened skin with slightly raised plaques, papules, or confluent papules; and/or with biopsies showing increased numbers of fibroblasts, alteration of the normal pattern of collagen bundles seen in the dermis, and increased dermal deposits of mucin.

In some embodiments, a dermal fibrosing syndrome is associated with proliferation and/or activation of fibroblasts in any dermal layer. In some embodiments, administration of one or more wortmannin analogs and/or PI-3 kinase inhibitors (e.g., compounds of Formula IA, IB, IIA or IIB) to an individual in need thereof inhibits or partially inhibits proliferation of fibroblasts, thereby reducing, reversing, or delaying progression and/or onset of any dermal fibrosing syndrome described herein.

Fibrosing Syndromes Associated with Organ Transplant

In some embodiments, a fibrosing syndrome is associated with organ transplant (including allograft and/or xenograft) such as liver allograft (e.g., fibrosing cholestatic hepatitis, liver fibrosis, kidney fibrosis or the like). In some embodiments, administration of one or more wortmannin analogs and/or PI-3 kinase inhibitors (e.g., compounds of Formula IA, IB, IIA or IIB) to an individual in need thereof reduces or prevents the occurrence of fibrosis in a transplanted organ or in the vicinity of a transplanted organ.

### Other Fibrosing Syndromes

Provided herein are methods of treatment of certain other fibrosing syndromes comprising administration of one or more wortmannin analogs and/or inhibitors of PI-3 kinases (e.g., compounds of Formula IA, IB, IIA or IIB) to an individual in need thereof. Such fibrosing syndromes include and are not limited to cystic fibrosis, endomyocardial fibrosis, mediastinal fibrosis, myelofibrosis, osteofibrosis, multifocal fibrosclerosis, anaphylactic shock fibrosis, or the like.

Examples 3-12 describe the use of certain wortmannin analogs and/or PI-3 kinase inhibitors (e.g., compounds of Formula IA, IB, IIA or IIB, or any other PI-3 kinase inhibitor described herein) for treatment of certain fibrosing syndromes described above.

In some embodiments, any fibrosing syndrome described above is associated with proliferation and/or activation of fibroblasts. In some embodiments, administration of one or more wortmannin analogs and/or PI-3 kinase inhibitors (e.g., compounds of Formula IA, IB, IIA or IIB, or any other PI-3 kinase inhibitor described herein) to an individual in need thereof inhibits or partially inhibits proliferation of fibroblasts, thereby reducing, reversing, or delaying progression and/or onset of any fibrosing syndrome described above.

In one embodiment, provided herein is a method of treating any fibrosing syndrome (e.g., pulmonary fibrosis) described above in a subject comprising administering to a subject a therapeutically effective amount of PX-866 having a structure of:

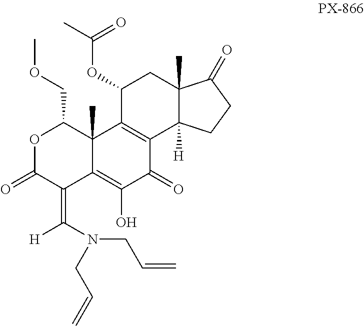

In one embodiment provided herein is a method of treating any fibrosing syndrome (e.g., pulmonary fibrosis) described above in a subject in need thereof comprising administering to a subject a therapeutically effective amount of PX-867 having a structure of:

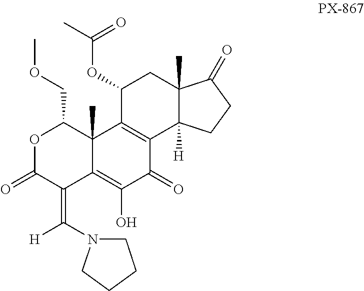

Certain further embodiments provide for methods of treating a fibrosing syndrome (e.g., pulmonary fibrosis) comprising administration of a therapeutically effective amount of wortmannin analog and metabolites selected from, but not limited to, PX-868, PX-870, PX-871, PX-880, PX-881, PX-882, PX-889, PX-890, DJM2-170, DJM2-171, DJM2-177, DJM2-181 and combinations thereof to an individual in need thereof.

### Combination Therapy

In some embodiments, provided herein are methods for the use of PI-3 kinase inhibitors (e.g., compounds of Formula IA, IB, IIA or IIB, or any other PI-3 kinase inhibitor described herein) in combination with secondary therapeutic agents to treat fibrosing syndromes (e.g., pulmonary fibrosis).

In some embodiments, provided herein are methods for the use of wortmannin analogs or metabolites in combination with secondary therapeutic agents to treat fibrosing syndromes (e.g., pulmonary fibrosis).

Examples of secondary therapeutic agents include and are not limited to immune-suppressants such as, for example, corticosteroids (e.g., prednisone, dexamethasone, triamcinolone or any other corticosteroid), gamma-interferon, Serum Amyloid P, cyclophosphamide, azathioprine, methotrexate, penicillamine, cyclosporine or the like. Other secondary therapeutic agents include colchicine, mycophenolate mofetil, perfenidone or the like. In some embodiments, secondary therapeutic agents are protein therapeutic agents (e.g., antibodies).

The mammalian target of rapamycin (mTOR) is a highly conserved intracellular serine/threonine kinase and a major downstream component in the PI3K pathway. Certain studies demonstrate that the PI3K-Akt-mTOR pathway mediates the fibrotic response induced by EGFR activation in the lung. Accordingly, in some embodiments, methods of treatment of fibrosis described herein comprise administration of small molecule EGFR tyrosine kinase inhibitors (e.g., gefitinib, erlotinib or the like) in combination with PI-3 kinase inhibitors for prevention, delayed progression, reversal and/or partial reversal of established pulmonary fibrosis and/or any other fibrotic condition described herein. In some embodiments, methods of treatment of fibrosis described herein comprise administration of small molecule mTor inhibitors including and not limited to rapamycin, Temsirolimus, Deforolimus, Everolimus, BEZ235 or the like for prevention, delayed progression, reversal and/or partial reversal of established pulmonary fibrosis and/or any other fibrotic condition described herein.

### Pharmaceutical Composition/Formulation

In some embodiments, the compounds described herein are formulated into pharmaceutical compositions. In specific embodiments, pharmaceutical compositions are formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising a compound of Formula IA, IB, IIA or IIB or any other PI-3 kinase inhibitor and/or wortmannin analog described herein and a pharmaceutically acceptable diluent(s), excipient(s), or carrier(s). In certain embodiments, the compounds described herein are administered as pharmaceutical compositions in which a compound of Formula IA, IB, IIA or IIB or any other PI-3 kinase inhibitor and/or wortmannin analog described herein is mixed with other active ingredients, as in combination therapy. Encompassed herein are all combinations of actives set forth in the combination therapies section below and throughout this disclosure. In specific embodiments, the pharmaceutical compositions include one or more compounds of Formula IA, IB, IIA or IIB.

A pharmaceutical composition, as used herein, refers to a mixture of a compound of Formula IA, IB, IIA or IIB or any other PI-3 kinase inhibitor and/or wortmannin analog described herein with other chemical components, such as carriers, stabilizers, diluents, dispersing agents, suspending agents, thickening agents, and/or excipients. In certain embodiments, the pharmaceutical composition facilitates administration of the compound to an organism. In some embodiments, practicing the methods of treatment or use provided herein, therapeutically effective amounts of compounds of Formula IA, IB, IIA or IIB or any other PI-3 kinase inhibitor and/or wortmannin analog described herein are administered in a pharmaceutical composition to a mammal having a disease or condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In such a composition, the pharmacologically active compound is known as the “active ingredient”. In making the compositions, the active ingredient will usually be mixed with a carrier, or diluted by a carrier, or enclosed within a carrier that may be in the form of a capsule, sachet, paper or other container. When the carrier serves as a diluent, it may be a solid, semisolid, or liquid material that acts as a vehicle, excipient of medium for the active ingredient. Thus, the composition can be in the form of tablets, pills, powders, lozenges, sachets, cachets, elixirs, emulsions, solutions, syrups, suspensions, soft and hard gelatin capsules, sterile injectable solutions, and sterile packaged powders.

Some examples of suitable carriers, excipients, and diluents include lactose, dextrose, sucrose, sorbitol, mannitol, starches, gum acacia, calcium phosphate alginates, calcium salicate, microcrystalline cellulose, polyvinylpyrrolidone, cellulose, tragacanth, gelatin, syrup, methyl cellulose, methyl- and propylhydroxybenzoates, talc, magnesium stearate, water, and mineral oil. The compositions can additionally include lubricating agents, wetting agents, emulsifying and suspending agents, preserving agents, sweetening agents or flavoring agents. The compositions may be formulated so as to provide quick, sustained, or delayed release of the active ingredient after administration to the patient by employing procedures well known in the art.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation.

The local delivery of inhibitory amounts of an active compound for the treatment of a fibrosis (e.g., pulmonary fibrosis) can be by a variety of techniques that administer the compound at or near the fibrotic site. Examples of local delivery techniques are not intended to be limiting but to be illustrative of the techniques available. Examples include local delivery catheters, site specific carriers, implants, direct injection, or direct applications. Local delivery by a catheter allows the administration of a therapeutic agent directly to the fibrotic site.

Local delivery by an implant describes the surgical placement of a matrix that contains the therapeutic agent into the fibrotic organ (e.g., lung(s)). The implanted matrix releases the therapeutic agent by diffusion, chemical reaction, or solvent activators.

Another example is the delivery of a therapeutic agent by polymeric endoluminal sealing. This technique uses a catheter to apply a polymeric implant to the interior surface of the lumen. The therapeutic agent incorporated into the biodegradable polymer implant is thereby released at the surgical site. It is described in PCT WO 90/01969 (Schindler, Aug. 23, 1989).

A further example of local delivery by an implant is by direct injection of vesicles or microparticulates into the site. These microparticulates may be composed of substances such as proteins, lipids, carbohydrates or synthetic polymers. These microparticulates have the therapeutic agent incorporated throughout the microparticle or over the microparticle as a coating. Delivery systems incorporating microparticulates are described in Lange, Science 249:1527-1533 (1990) and Mathiowitz et al, J. App. Poly. Sci., 26:809 (1981).

Local delivery by site specific carriers describes attaching the therapeutic agent to a carrier which will direct the drug to the target fibrotic organ (e.g, lung(s)). Examples of this delivery technique include the use of carriers such as a protein ligand or a monoclonal antibody.

In some embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

In one embodiment, one or more compounds of Formula IA, IB, IIA or IIB or any other PI-3 kinase inhibitor and/or wortmannin analog described herein is formulated in an aqueous solution. In specific embodiments, the aqueous solution is selected from, by way of example only, a physiologically compatible buffer, such as Hank's solution, Ringer's solution, or physiological saline buffer. In other embodiments, one or more compound of Formula IA, IB, IIA or IIB or any other PI-3 kinase inhibitor and/or wortmannin analog described herein is formulated for transmucosal administration. In specific embodiments, transmucosal formulations include penetrants that are appropriate to the barrier to be permeated. In still other embodiments wherein the compounds described herein are formulated for other parenteral injections, appropriate formulations include aqueous or nonaqueous solutions. In specific embodiments, such solutions include physiologically compatible buffers and/or excipients.

In another embodiment, compounds described herein are formulated for oral administration. Compounds described herein, including compounds of Formula IA, IB, IIA or IIB or any other PI-3 kinase inhibitor and/or wortmannin analog described herein, are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

For oral administration, a compound can be admixed with carriers and diluents, molded into tablets, or enclosed in gelatin capsules.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In other embodiments, therapeutically effective amounts of at least one of the compounds described herein are formulated for buccal or sublingual administration. Formulations suitable for buccal or sublingual administration include, by way of example only, tablets, lozenges, or gels.

In still other embodiments, the compounds described herein are formulated for parental injection, including formulations suitable for bolus injection or continuous infusion. The compounds of Formula IA, IB, IIA or IIB or any other PI-3 kinase inhibitor and/or wortmannin analog described herein can alternatively be dissolved in liquids such as 10% aqueous glucose solution, isotonic saline, sterile water, or the like, and administered intravenously or by injection.

In specific embodiments, formulations for injection are presented in unit dosage form (e.g., in ampoules) or in multi-dose containers. Preservatives are, optionally, added to the injection formulations. In still other embodiments, the pharmaceutical composition of Formula IA, IB, IIA or IIB or any other PI-3 kinase inhibitor and/or wortmannin analog described herein are formulated in a form suitable for parenteral injection as a sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of the active compounds are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

In one aspect, compounds of Formula IA, IB, IIA or IIB or any other PI-3 kinase inhibitor and/or wortmannin analog described herein are prepared as solutions for parenteral injection as described herein or known in the art and administered with an automatic injector. Automatic injectors, such as those disclosed in U.S. Pat. Nos. 4,031,893, 5,358,489; 5,540,664; 5,665,071, 5,695,472 and WO/2005/087297 (each of which are incorporated herein by reference for such disclosure) are known. In general, all automatic injectors contain a volume of solution that includes a compound of Formula IA, IB, IIA or IIB or any other PI-3 kinase inhibitor and/or wortmannin analog described herein to be injected. In general, automatic injectors include a reservoir for holding the solution, which is in fluid communication with a needle for delivering the drug, as well as a mechanism for automatically deploying the needle, inserting the needle into the patient and delivering the dose into the patient. Exemplary injectors provide about 0.3 mL of solution at about a concentration of 0.5 mg to 10 mg of compound of Formula IA, IB, IIA or IIB or any other PI-3 kinase inhibitor and/or wortmannin analog described herein per 1 mL of solution. Each injector is capable of delivering only one dose of the compound.

In still other embodiments, the compounds of Formula IA, IB, IIA or IIB or any other PI-3 kinase inhibitor and/or wortmannin analog described herein are administered topically. The compounds described herein are formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In yet other embodiments, the compounds of Formula IA, IB, IIA or IIB or any other PI-3 kinase inhibitor and/or wortmannin analog described herein are formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of the compounds of Formula IA, IB, IIA or IIB or any other PI-3 kinase inhibitor and/or wortmannin analog described herein is accomplished by means of iontophoretic patches and the like. In certain embodiments, transdermal patches provide controlled delivery of the compounds of Formula IA, IB, IIA or IIB or any other PI-3 kinase inhibitor and/or wortmannin analog described herein. In specific embodiments, the rate of absorption is slowed by using rate-controlling membranes or by trapping the compound within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

Transdermal formulations described herein may be administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

The transdermal dosage forms described herein may incorporate certain pharmaceutically acceptable excipients which are conventional in the art. In one embodiment, the transdermal formulations described herein include at least three components: (1) a formulation of a compound of Formula IA, IB, IIA or IIB or any other PI-3 kinase inhibitor and/or wortmannin analog described herein; (2) a penetration enhancer; and (3) an aqueous adjuvant. In addition, transdermal formulations can include additional components such as, but not limited to, gelling agents, creams and ointment bases, and the like. In some embodiments, the transdermal formulation further include a woven or non-woven backing material to enhance absorption and prevent the removal of the transdermal formulation from the skin. In other embodiments, the transdermal formulations described herein maintain a saturated or supersaturated state to promote diffusion into the skin.

In other embodiments, the compounds of Formula IA, IB, IIA or IIB or any other PI-3 kinase inhibitor and/or wortmannin analog described herein are formulated for administration by inhalation. Various forms suitable for administration by inhalation include, but are not limited to, aerosols, mists or powders. Pharmaceutical compositions of Formula IA, IB, IIA or IIB or any other PI-3 kinase inhibitor and/or wortmannin analog described herein are conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatin for use in an inhaler or insufflator are formulated containing a powder mix of the compound and a suitable powder base such as lactose or starch.

Intranasal formulations are known in the art and are described in, for example, U.S. Pat. Nos. 4,476,116, 5,116,817 and 6,391,452, each of which is specifically incorporated by reference. Formulations, which include a compound of Formula IA, IB, IIA or IIB or any other PI-3 kinase inhibitor and/or wortmannin analog described herein, which are prepared according to these and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are found in sources such as REMINGTON: THE SCIENCE AND PRACTICE OF PHARMACY, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. Preferably, the nasal dosage form should be isotonic with nasal secretions.

For administration by inhalation, the compounds described herein, may be in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

In still other embodiments, the compounds of Formula IA, IB, IIA or IIB or any other PI-3 kinase inhibitor and/or wortmannin analog described herein are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In certain embodiments, pharmaceutical compositions are formulated in any conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients is optionally used as suitable and as understood in the art. Pharmaceutical compositions comprising a compound of Formula IA, IB, IIA or IIB or any other PI-3 kinase inhibitor and/or wortmannin analog described herein may be manufactured in a conventional manner, such as, by way of example only, by means of conventional mixing, dissolving, granulating, dragee-making, levigating, emulsifying, encapsulating, entrapping or compression processes.

Pharmaceutical compositions include at least one pharmaceutically acceptable carrier, diluent or excipient and at least one compound of Formula IA, IB, IIA or IIB or any other PI-3 kinase inhibitor and/or wortmannin analog described herein as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical composition comprising at least one compound of Formula IA, IB, IIA or IIB or any other PI-3 kinase inhibitor and/or wortmannin analog described herein illustratively takes the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, pharmaceutical aqueous suspensions include one or more polymers as suspending agents. Polymers include water-soluble polymers such as cellulosic polymers, e.g., hydroxypropyl methylcellulose, and water-insoluble polymers such as cross-linked carboxyl-containing polymers. Certain pharmaceutical compositions described herein include a mucoadhesive polymer, selected from, for example, carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally include solubilizing agents to aid in the solubility of a compound of Formula IA, IB, IIA or IIB or any other PI-3 kinase inhibitor and/or wortmannin analog described herein. The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other pharmaceutical compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other pharmaceutical compositions may include one or more antioxidants to enhance chemical stability where required. Suitable antioxidants include, by way of example only, ascorbic acid and sodium metabisulfite.

In certain embodiments, pharmaceutical aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the compounds for a few hours up to over 24 hours. Depending on the chemical nature and the biological stability of the therapeutic reagent, additional strategies for protein stabilization may be employed.

In certain embodiments, the formulations described herein include one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

### Methods of Dosing and Treatment Regimens

In one embodiment, the compound of Formula IA, IB, IIA or IIB or any other PI-3 kinase inhibitor and/or wortmannin analog described herein are used in the preparation of medicaments for the treatment of fibrotic conditions. In addition, a method for treating any of the diseases or conditions described herein in a subject in need of such treatment, involves administration of pharmaceutical compositions containing at least one compound of Formula IA, IB, IIA or IIB or any other PI-3 kinase inhibitor and/or wortmannin analog described herein, or a pharmaceutically acceptable salt, pharmaceutically active metabolite, pharmaceutically acceptable prodrug, or pharmaceutically acceptable solvate thereof, in therapeutically effective amounts to said subject.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered may be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but can nevertheless be determined according to the particular circumstances surrounding the case, including, e.g., the specific agent being administered, the route of administration, the condition being treated, and the subject or host being treated. In general, however, doses employed for adult human treatment are typically in the range of 0.02 mg-5000 mg per day, preferably 1-1500 mg per day. In one embodiment, the desired dose is conveniently presented in a single dose or in divided doses administered simultaneously (or over a short period of time) or at appropriate intervals, for example as two, three, four or more sub-doses per day.

In some embodiments, compounds of Formula IA, IB, IIA or IIB or any other PI-3 kinase inhibitor and/or wortmannin analog described herein are administered chronically. In some embodiments, compounds of Formula IA, IB, IIA or IIB or any other PI-3 kinase inhibitor and/or wortmannin analog described herein are administered intermittently (e.g. drug holiday that includes a period of time in which the compound is not administered or is administered in a reduced amount). In some embodiments, compounds of Formula IA, IB, IIA or IIB or any other PI-3 kinase inhibitor and/or wortmannin analog described herein are administered in cycles that include: (a) a first period that includes daily administration of the compound of Formula IA, IB, IIA or IIB or any other PI-3 kinase inhibitor and/or wortmannin analog described herein; followed by (b) a second period that includes a dose reduction of the daily amount of the compound of Formula IA, IB, IIA or IIB or any other PI-3 kinase inhibitor and/or wortmannin analog described herein that is administered. In some embodiments, the compound of Formula IA, IB, IIA or IIB or any other PI-3 kinase inhibitor and/or wortmannin analog described herein is not administered in the second period. In some embodiments, the duration of the first and second periods, as well as the dose amounts are determined using methods described herein or known in the art. In some instances, a drug holiday or a dose reduction period is appropriate depending on the pharmacodynamic profile of the active agent.

In certain embodiments, the pharmaceutical composition described herein is in unit dosage forms suitable for single administration of precise dosages. In unit dosage form, the formulation is divided into unit doses containing appropriate quantities of one or more compound. In specific embodiments, the unit dosage is in the form of a package containing discrete quantities of the formulation. Non-limiting examples are packaged tablets or capsules, and powders in vials or ampoules. Aqueous suspension compositions are optionally packaged in single-dose non-re-closeable containers. Alternatively, multiple-dose re-closeable containers are used, in which case it is typical to include a preservative in the composition. By way of example only, formulations for parenteral injection are, in some embodiments, presented in unit dosage form, which include, but are not limited to ampoules, or in multi-dose containers, with an added preservative.

In one embodiment, the daily dosages appropriate for the compound of Formula IA, IB, IIA or IIB or any other PI-3 kinase inhibitor and/or wortmannin analog described herein are from about 0.001 to about 100 mg/kg per body weight. In one embodiment, the daily dosages appropriate for the compound of Formula IA, IB, IIA or IIB or any other PI-3 kinase inhibitor and/or wortmannin analog described herein are from about 0.01 to about 10 mg/kg per body weight. In some embodiments, an indicated daily dosage in a large mammal, including, but not limited to, humans, is in the range from about 0.5 mg to about 1000 mg, conveniently administered in divided doses, including, but not limited to, up to four times a day. In one embodiment, the daily dosage is administered in extended release form. In certain embodiments, suitable unit dosage forms for oral administration comprise from about 1 to 500 mg active ingredient. In other embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

This invention and embodiments illustrating the method and materials used may be further understood by reference to the following non-limiting examples.

### EXAMPLES

### Example 1

### Transgenic Mice and Administration of PX-866

CCSP-rtTA activator mice expressing the reverse tetracycline-responsive transactivator (rtTA) under control of the 2.3-kb rat Clara Cell Secretory Protein (CCSP), a.k.a. secretoglobin, family 1A, member 1 (Scgblal) gene promoter were mated to conditional doxycycline (Dox) regulated transgenic mice containing the human TGFα cDNA under the control of seven copies of the tetracycline operon ((TetO)₇-cmv TGFα) plus a minimal CMV promoter. Single transgenic (CCSP-rtTA+/−) and bitransgenic CCSP-rtTA+/−/(TetO)₇-cmv TGFα+/− mice were produced within the same litter by mating homozygous CCSP-rtTA+/+ mice to hemizygous (TetO)?-cmv TGFα+/− mice. All mice were derived from the FVB/NJ inbred strain. Mice were maintained in virus-free containment. All animal protocols were reviewed and approved by the Institutional Animal Use and Care Committee of the Cincinnati Children's Hospital Research Foundation. To induce TGFα expression, Dox (Sigma, St. Louis, Mo.) was administered in the drinking water at a final concentration of 0.5 mg/ml and in food (62.5 mg/kg). Water was replaced three times per week. Mice were genotyped as known in the art.

The PI3K inhibitor PX-866 (ProIX Pharmaceuticals, Tucson, Ariz.) was suspended into 5% EtOH to make a 5 mg/ml stock solution. Three hours prior to administration, food and water were removed from cages. Mice were then anesthetized (Isoflurane; Abbott Labs, Chicago, Ill.), and sterile vehicle or drug (3 mg/kg) was administered by gavage using a 20 gauge feeding catheter (Harvard Apparatus, Holliston, Mass.). Mice were treated with vehicle or PX-866 every other day for up to 4 weeks. Mice were killed with pentobarbital sodium (65 mg/ml) euthanasia solution (Fort Dodge Animal Health, Fort Dodge, Iowa) 1 day or 4 weeks after Dox and vehicle or PX-866 treatment.

Western Blots: Western blot analysis was performed on lung homogenates as previously described. Blots were incubated with antibodies against total and phosphorylated Akt (Ser 473 and Thr 308, Cell Signaling Technology) and quantified using the volume integration function on a PhosphorImager software Imagequant 5.2 (Molecular Dynamics, Sunnyvale, Calif.).

Lung Histology, Immunostaining and Total Lung Collagen: Lungs were inflation fixed as previously described. Sections (5 wm) were loaded onto polysine slides for trichrome staining as previously described. Total lung collagen was determined by quantifying total soluble collagen (Sircol Collagen Assay, Biocolor, Ireland) as previously described.

Pulmonary Mechanics: Lung mechanics were assessed on mice with a computerized Flexi Vent system (SCIREQ, Montreal, Canada). Mice were anesthetized with ketamine and xylazine, tracheostomized and then ventilated with a tidal volume of 8 ml/kg at a rate of 450 breaths/min and positive end-expiratory pressure (PEEP) of 2 cm H2O computerized by the SCIREQ system thereby permitting analysis of dynamic lung compliance. The ventilation mode was changed to forced oscillatory signal (0.5-19.6 Hz), and respiratory impedance was measured. Tissue elastance was obtained for mice at 2 cm H2O PEEP by fitting a model to each impedance spectrum. With this system, the calibration procedure removed the impedance of the equipment and tracheal tube.

PX-866 inhibits TGFα-Induced Phosphorylation of Akt: CCSP-rtTA/otet-TGFαmice were treated with 1 day of Dox to induce TGFα expression. Phosphorylated Akt (P-Akt) levels for Ser 473 as measured by Western blot analysis increased over 5-fold compared to Dox-treated control mice. P-Akt for Thr 308 did not change following TGFα expression (data not shown). PX-866 treatment in CCSP-rtTA/otet-TGFα mice prevented TGFα-induced increases in P-Akt (FIGS. 2A and 2B).

Statistics: Means (+/−SEM) were calculated and plotted for each variable, by mouse group (CCSP/-Vehicle, CCSP/TGFα Vehicle, CCSP/TGFα PX-866). Data were assessed for normality using plots and the Shapiro-Wilk test. Where normality assumptions were not met, log-transformed values were used in a one-way ANOVA to test for differences between groups. Where log-transformations did not improve normality, a non-parametric, one-way ANOVA was used. Simulation based, step-down multiple comparison adjustments were used for all pair-wise comparisons. For the non-parametric ANOVAs, the Bonferroni-Holm multiple comparison adjustment was used.

Weights of mice in four different groups were measured at Baseline and at evenly spaced intervals during the next eight weeks, once per week. A repeated measures analysis was conducted with Group*Time and Baseline as the factors, in order to compute differences in Group*Time means. A separate Toepliz variance/covariance structure was used for each Group. Differences in selected (a priori) Group*Time means were calculated and tested using a simulation-based adjustment for multiple comparisons.

### Example 2

### PX-866 Inhibits TGFα-Induced Pulmonary Fibrosis

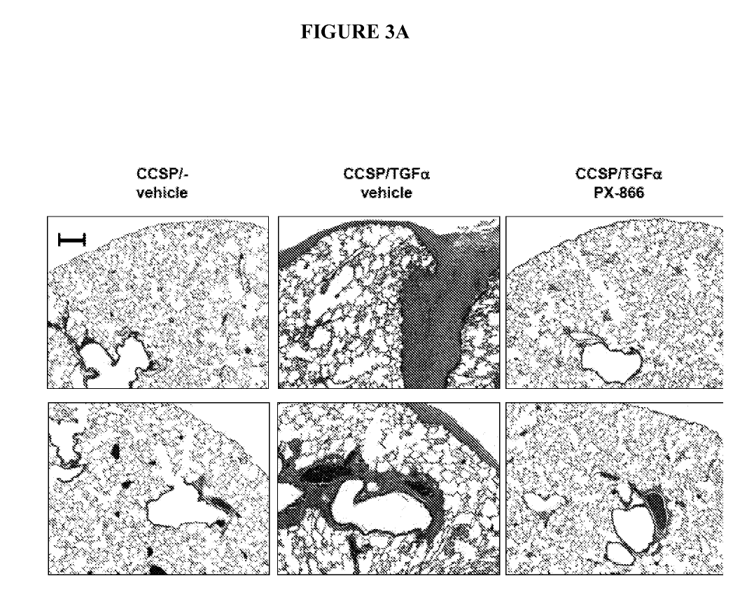

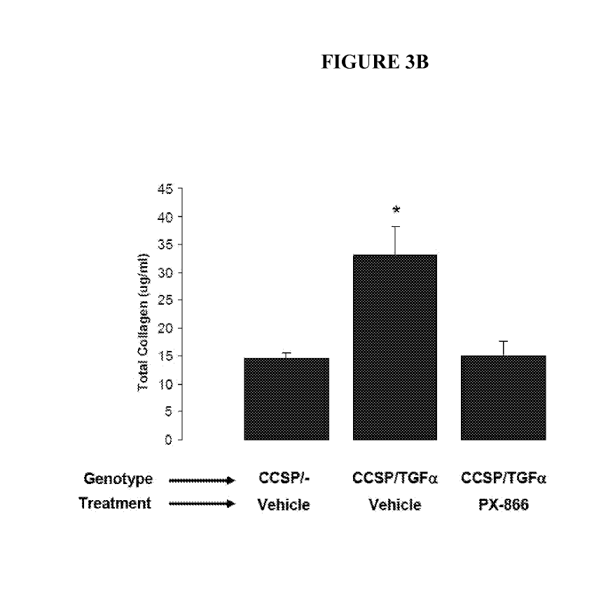

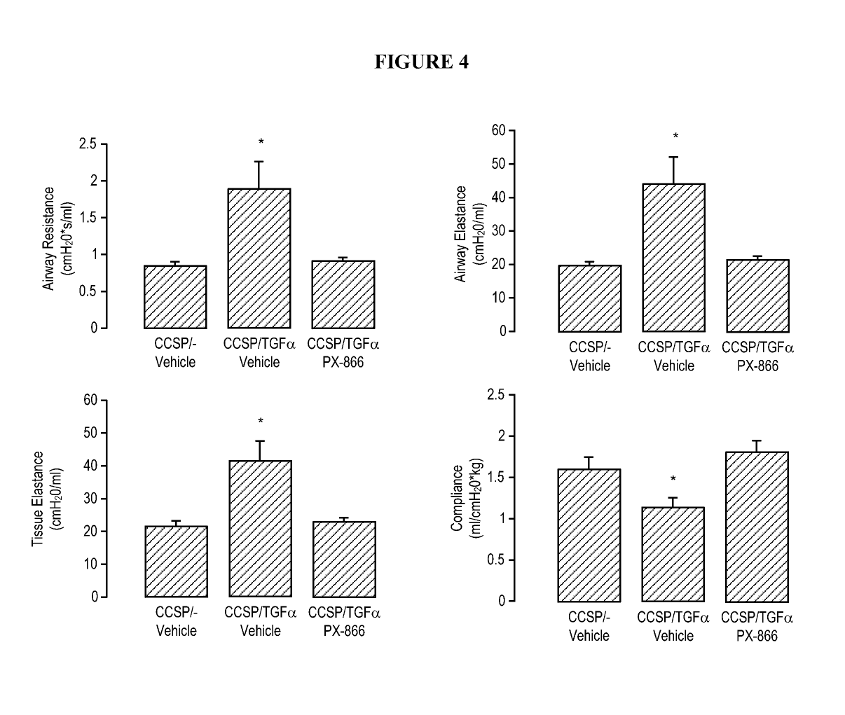

CCSP-rtTA/otet-TGFα mice were treated with Dox to induce TGFα expression and concomitantly treated with either PX-866 (4 mg/kg every other day) or vehicle for 4 weeks. Induction of TGFα caused extensive pleural, perivascular and peribronchial fibrosis (FIG. 3A). Total lung collagen levels were over 2-fold higher in CCSP-rtTA/otet-TGFα mice compared to Dox-treated control mice. Mice treated with PX-866 did not show any differences in lung fibrosis as assessed by histology and whole lung collagen compared to Dox-treated control mice (FIGS. 3A and 3B). Lung compliance decreased by more than 30%, and airway resistance, elastance and tissue elastance increased more than 2-fold in CCSP-rtTA/otet-TGFα mice compared to Dox-treated control mice. Mice treated with PX-866 did not show any differences in lung mechanics compared to Dox-treated control mice (FIG. 4).

### PX-866 Prevents Progression of Established TGFα-Induced Pulmonary Fibrosis:

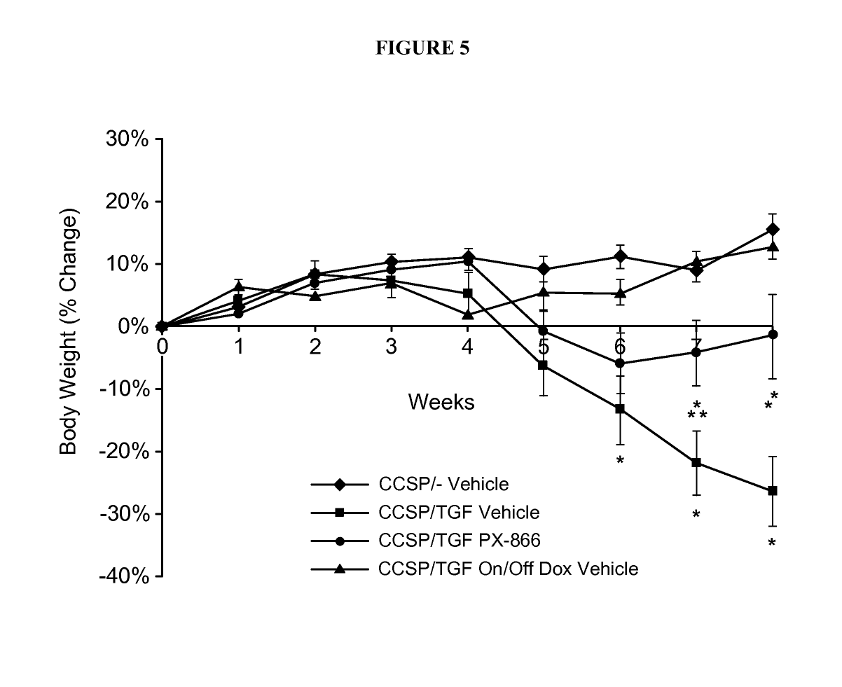

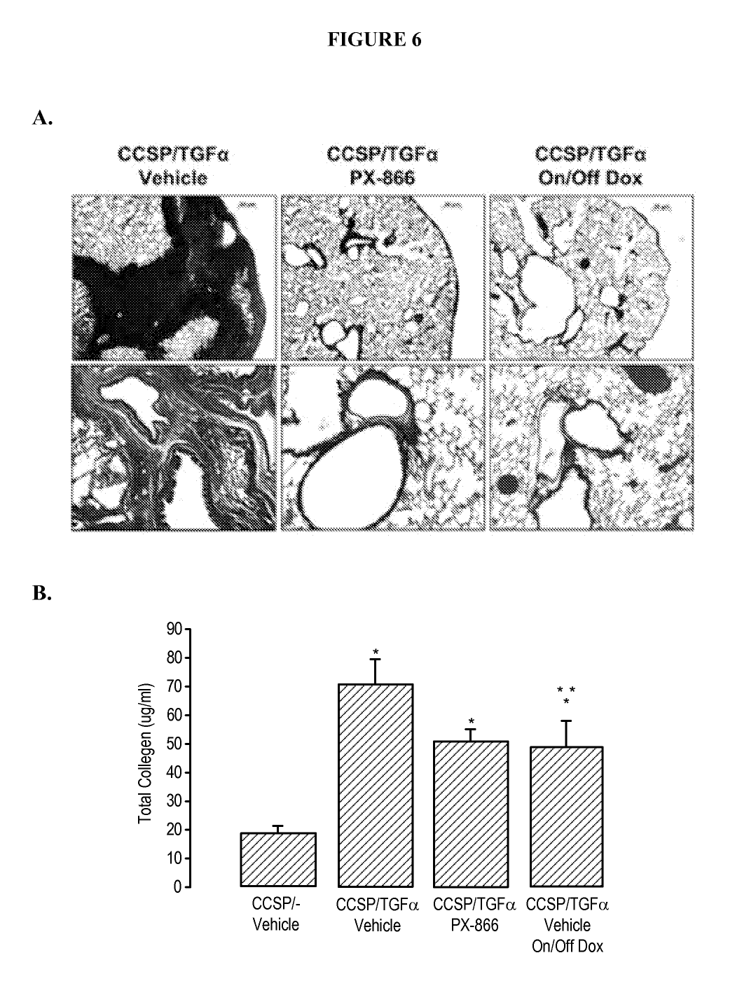

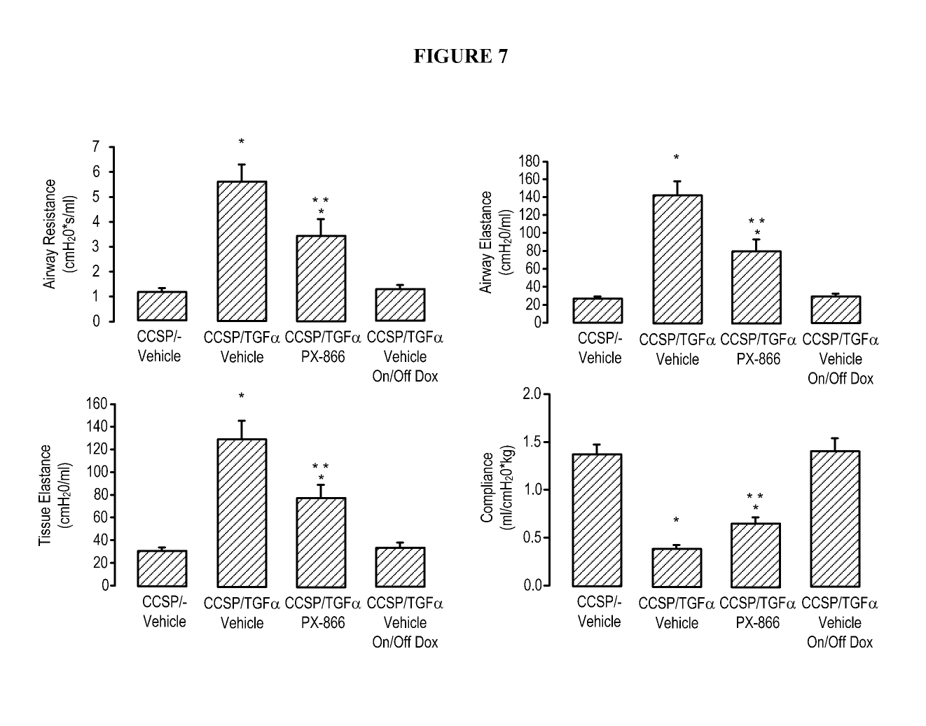

To determine whether PX-866 influences the progression of established fibrosis, following 4 weeks of Dox treatment, CCSP-rtTA/otet-TGFα mice were administered PX-866 while remaining on Dox for an additional 4 weeks (8 weeks total). Controls included CCSP/- and CCSP-rtTA/otet-TGFα mice treated with vehicle while remaining on Dox an additional 4 weeks. A third set of controls included CCSP-rtTA/otet-TGFα mice which received 4 weeks of Dox, then taken off Dox and treated with 4 weeks of vehicle (FIG. 5A). The on-off Dox group is added to compare the efficacy of PX-866 in reversing fibrosis in mice with ongoing EGFR activation to mice where EGFR activation is extinguished. Body weights of CCSP-rtTA/otet-TGFα mice treated with vehicle decreased over 26% from baseline following 8 weeks of Dox (FIG. 5B). PX-866 administered at the beginning of week 5 prevented further body weight loss compared to vehicle-treated CCSP-rtTA/otet-TGFα mice, but body weights remained less than CCSP/-control mice or CCSP-rtTA/otet-TGFα mice off Dox. CCSP-rtTA/otet-TGFα mice treated with Dox and vehicle for 8 weeks demonstrated marked pleural thickening with fibrosis advancing into the interstitium and effacing alveolar architecture (FIG. 6A). In addition there was advanced perivascular and peribronchial fibrosis in large and small vessels and airways. CCSP-rtTA/otet-TGFα mice treated with PX-866 demonstrated reduced pleural fibrosis as well as reduced perivascular and peribronchial fibrosis compared with vehicle-treated mice. CCSP-rtTA/otet-TGFα mice off Dox also demonstrated similar reduced pleural and adventitial fibrosis with little fibrosis seen in small airways and vessels. Total lung collagen levels were almost 4-fold higher in CCSP-rtTA/otet-TGFα mice compared to Dox-treated CCSP/-control mice after 8 weeks of Dox (FIG. 6B). Both CCSP-rtTA/otet-TGFα mice treated with PX-866 and mice off Dox demonstrated reduced lung collagen levels compared to vehicle-treated mice, but levels remained significantly elevated compared to CCSP/-control mice. Lung mechanics of CCSP-rtTA/otet-TGFα mice treated with PX-866 were significantly improved compared with vehicle-treated mice, but also remained significantly altered compared with controls and CCSP-rtTA/otet-TGFα mice off Dox for 4 weeks (FIG. 7).

To more closely mirror clinical treatment of individuals with lung fibrosis, the role of PI3K signaling is determined for maintenance of established TGFα-induced fibrosis. Mice treated with PX-866 4 weeks into Dox demonstrated normalization of body weights, reduced fibrosis on lung histology and improved lung mechanics compared with vehicle-treated mice. However, body weights, lung histology and collagen and lung mechanics all remained altered compared with CCSP/-control mice demonstrating incomplete reversal of the fibrosis phenotype. As the fibrotic process may not be expected to be completely resolved 4 weeks into treatment, endpoints were compared in mice where TGFα over-expression was extinguished by removing Dox. If fibrosis endpoints were similar between the PX-866 and Off Dox groups, PI3K inhibition is likely to be effective in reversing lung fibrosis. Present study demonstrates that PX-866 treated mice showed similar degrees of fibrosis measured by lung collagen and histology compared to mice Off Dox, while physiologic measures of fibrosis including body weights and lung mechanics remained altered in PX-866-treated mice. To further assess PI3K inhibition in reversing fibrosis, PX-866-treated mice were compared to mice after only 4 weeks of Dox. Fibrosis endpoints in the 4 week Dox group (FIGS. 3 and 4) represent the new starting point of lung fibrosis when mice begin treatment. If PI3K inhibition reverses fibrosis, fibrosis endpoints are expected to improve compared to 4 weeks Dox mice. PX-866-treated mice demonstrated similar degrees of fibrosis measured by lung histology and collagen compared to 4 week Dox mice, while lung mechanics remained significantly altered in PX-866-treated mice. Taken together, reversal studies demonstrate that PI3K inhibition, after fibrosis is established, prevented progression of lung fibrosis but attenuated physiologic alterations. The weekly body weigh values in the PX-866-treated mice trended upward for the final 2 weeks of treatment suggesting a delayed recovery and resolution of fibrosis in PX-866 treated mice.

### Example 3

### Bleomycin Induced Mouse Lung Fibrosis Model

A mouse model of drug-induced lung fibrosis is used in this study. The protocol is adapted from the protocol described by Walters et al. in Current Protocols in Pharmacology, posted online March 2008. Bleomycin is delivered either directly into the lung or systemically, to create models of lung fibrosis in mice. Formulations comprising PX-866 or PX-867 are administered therapeutically or prophylactically. Lung collagen content is determined using a Sircol Soluble Collagen Assay (Biocolor, Ltd.; available from Accurate Chemical and Scientific). A reduction of collagen content in the lung is indicative of a therapeutic effect in this model.

### Example 4

### Animal Model for Intestinal Fibrosis

A murine model of chronic intestinal fibrosis described in Gastroenterology 2003, 125, 1750-61 is used in this study. Chronic inflammation is established by weekly injections of trinitrobenzene sulfonic acid (TNBS). Fibrosis typically persists for 2-4 weeks after cessation of TNBS injections. A formulation comprising PX-866 is administered therapeutically or prophylactically.

Colonic fibrosis is determined by histology. Total collagen level is examined by hydroxyproline quantification as described by Kivirikko et al., Anal Biochem. 1967 19:249-55. Control and TNBS-treated colonic mesenchymal cells are characterized by morphology and phenotype. Colonic expression of transforming growth factor beta-1 (TGF-β-1) is determined by semiquantitative polymerase chain reaction. A reduction in collagen levels and expression of TGF-β-1 is indicative of a therapeutic effect on intestinal fibrosis in this model.

### Example 5

### Rabbit Wound Healing and Hypertrophic Scar Model

Following anesthesia, ear wounds are created in 10 young adult female New Zealand rabbits, 4 wounds per ear on each ear for a total of 8 wounds per animal. Wounds are created using a 7-mm biopsy punch with the wound created to go to bare cartilage. A dissecting microscope is used to ensure complete removal of the epidermis, dermis and perichondrium in each wound. For the hypertrophic scar model, it is the removal of the perichondrial layer and subsequent delay in reepithelialization of the defect that results in the elevated scar. Each wound heals independently and is considered a separate sample.

Two treatment groups are examined to study the early phase and a later phase of wound healing. The early treatment group (n=15 rabbits, 120 wounds) are treated with either the test compound formulated as a 0.05-1.5% by weight topical formulation (solution, cream, ointment or gel) or placebo using the topical vehicle formulation post-wounding on days 0, 1, 2, 3, 4, 5, 6 and 7 and harvested on day 28 after wounding. The later treatment group (n=15 rabbits, 120 wounds) are treated with either the test compound formulated as a 0.05-1.5% topical formulation (solution, cream, ointment or gel) or placebo using the topical vehicle formulation post-wounding on days 7, 8, 9, 10, 11, 12, 13 and 14 and harvested on day 28 after wounding. Half of the wounds in each group are treated with active compound and half are treated with placebo. Each wound is covered with a sterile dressing (Tegaderm; 3M) and dressings are changed daily following each treatment and as needed until the wound appears reepithelialized on gross examination. Wounds are excluded from analysis if there is evidence of infection, desiccation or necrosis.

At the end of each study wounds are harvested with a 5-mm margin of surrounding unwounded tissue. The scars are bisected and half of each wound is fixed in 4% neutral-buffered formaldehyde, dehydrated, embedded in paraffin, cut in 4-μm sections, and stained with Masson's trichrome or sirrus red. The other half of each wound is flash frozen in liquid nitrogen and stored for RNA extraction

### Histologic Analysis

Light microscopy is used to examine each tissue section and the degree of wound healing and scar hypertrophy are measured with a calibrated lens reticle in a blinded fashion. Wound healing parameters: Relevant measurements are granulation tissue ingrowth volume and height, wound epithelialization, and wound closure. Each parameter is assessed twice and the results are averaged.

Scar hypertrophy parameters: The scar elevation index is determined as described by Lu et al, J. Am. Coll. Surg., 2005, 201, p391-397. The values are determined twice in a blinded fashion and the results averaged.

### Example 6

### Animal Model of Renal Fibrosis

Mice are sedated by general anesthesia, and an incision is made in the right side of the back. The right proximal ureter is exposed and double-ligated. Sham-operated mice have their ureter exposed but not ligated. The remodeling of the interstitium is then studied. Interstitial renal fibrosis is typically established about 15 days after surgery. Obstructed kidneys of mice showed fibrotic changes, with dilated renal tubules accompanied by proliferation of fibroblastic cells and influx of inflammatory mononuclear cells whereas normal architecture was preserved in sham-operated mice

An oral formulation of a compound of Formula IA, IB, IIA or IIB is administered therapeutically or prophylactically. Histomorphometric changes in the tubulointerstitial compartment are recorded using a Zeiss microscope equipped with a full colour 3CCD camera and KS-400 image analysis software from Zeiss-Kontron. Tissue is also observed for interstitial expression of smooth muscle alpha-actin. Accumulation of interstitial collagens is determined by immunoperoxidase and by Sirius red staining Reversal or reduction of fibrosis is indicative of therapeutic efficacy of oral doses of PX-866.

### Example 7

### Animal Model for Scarring Post Glaucoma Filtration Surgery

All experiments are performed with female chinchilla bastard rabbits (ChBB:CH), 3 to 6 months old and weighing 1.5 to 2.5 kg. Animals are acclimatized for 1 week before the experiments.

Surgery is performed on the right eye under general anesthesia with intramuscular injections of ketamine and xylazine and local anesthesia with oxybuprocaine drops. A peripheral iridectomy is performed as described in Grisanti et al. Investigative Ophthalmology and Visual Science, 2005; 46:191-196. On three consecutive days after surgery, each animal is administered once-a-day drops comprising PX-866 or PX-867.

Clinical examination is performed to evaluate the general appearance of the treated eyes, to assess local toxicity and ocular intolerance, and to measure the intraocular pressure. The loss of conjunctival transparency and thickening due to the deposition of fibrotic tissue is clinically examined to determine wound healing. Suppression of scarring is expected to maintain translucent conjunctiva.

All rabbits are killed on postoperative day 14, and the treated eyes are enucleated for histologic examination. Histologic analysis of the specimens is performed at the center of the sclerotomy site as indicated by the location of the iridectomy

The tissues are stained with hematoxylin and eosin to give an overall impression and with the Masson technique to determine the collagenous extracellular matrix (ECM) deposition. A reduction in ECM deposition is indicative of a therapeutic effect.

### Example 8

### Pharmaceutical Compositions

### Example 8a

### Parenteral Composition

To prepare a parenteral pharmaceutical composition suitable for administration by injection, 100 mg of a water-soluble salt of a compound described herein is dissolved in sterile water and then mixed with 10 mL of 0.9% sterile saline. The mixture is incorporated into a dosage unit form suitable for administration by injection.

### Example 8b

### Oral Composition

To prepare a pharmaceutical composition for oral delivery, 100 mg of a compound described herein is mixed with 750 mg of starch. The mixture is incorporated into an oral dosage unit for, such as a hard gelatin capsule, which is suitable for oral administration.

### Example 8c

### Sublingual (Hard Lozenge) Composition

To prepare a pharmaceutical composition for buccal delivery, such as a hard lozenge, mix 100 mg of a compound described herein, with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example 8d

### Inhalation Composition

To prepare a pharmaceutical composition for inhalation delivery, 20 mg of a compound described herein is mixed with 50 mg of anhydrous citric acid and 100 mL of 0.9% sodium chloride solution. The mixture is incorporated into an inhalation delivery unit, such as a nebulizer, which is suitable for inhalation administration.

### Example 8e

### Rectal Gel Composition

To prepare a pharmaceutical composition for rectal delivery, 100 mg of a compound described herein is mixed with 2.5 g of methylcelluose (1500 mPa), 100 mg of methylparapen, 5 g of glycerin and 100 mL of purified water. The resulting gel mixture is then incorporated into rectal delivery units, such as syringes, which are suitable for rectal administration.

### Example 8f

### Topical Gel Composition

To prepare a pharmaceutical topical gel composition, 100 mg of a compound described herein is mixed with 1.75 g of hydroxypropyl celluose, 10 mL of propylene glycol, 10 mL of isopropyl myristate and 100 mL of purified alcohol USP. The resulting gel mixture is then incorporated into containers, such as tubes, which are suitable for topical administration.

### Example 8g

### Ophthalmic Solution Composition

To prepare a pharmaceutical ophthalmic solution composition, 100 mg of a compound described herein is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

### Example 9

### Clinical Trial Evaluating Effect of a PI-3 Kinase Inhibitor Compound on the Treatment and Prevention of Recurrence of Excised Keloids. Treatment Commencing 7 Days Post-Surgery

A double blind, randomized, placebo controlled within trial study to evaluate the safety and efficacy of a compound administered topically following the excision of a keloid scar on the ear lobe. Each patient undergoes bilateral keloid scar excision and one ear lobe is treated with compound while the other ear lobe is treated with placebo such that each patient will act as their own control.

Ten to twelve subjects aged 18-65 years are to participate in the study. All subjects should have bilateral keloid scars suitable for surgical excision such that, following excision, the result will be a single wound on each ear lobe no greater than 2 cm long. The wound will be restricted to the skin, fat and fibrous tissue of the ear lobe.

No subject should have experienced keloid treatment with irradiation, cryosurgery, corticosteroids or other pharmacological agents within 12 weeks prior to the study. Subjects should not have a history of a bleeding disorder and should not have experienced or have on-going psoriasis or eczema or malignant skin tumors. Female subjects of child bearing potential must have a documented negative urine pregnancy test and must be practicing a medically proven form of contraception during the course of the study period. Written informed consent is obtained from each subject.

### Surgical Keloid Resection and Treatment Protocol

Ten to twelve patients will undergo bilateral keloid resection. Each patient will receive dermal administration of a PI-3 Kinase inhibitor compound formulated to an appropriate concentration of between 0.05 to 1.5% in a clinically acceptable and safe topical formulation (solution, cream, ointment or gel) to each linear centimeter of one ear lobe wound margin 7 days after wound closure and then repeatedly every 24 hours for 4 weeks. The other ear lobe will be treated topically with placebo (a clinically acceptable and safe topical formulation identical to that used in the treatment group, but lacking the active pharmaceutical ingredient) administered to each linear centimeter of ear lobe wound margin immediately after wound closure and then repeatedly every 24 hours for 4 weeks. The primary assessment is based on a photographic evaluation by a lay panel over a time period from week 4 to month 6 post surgery using a visual analog scale.

The primary outcome measure is to gain preliminary safety experience with the test compound in the keloid indication during the 52 week time frame. Secondary outcome measures are (i) reduction of keloid recurrence (Time frame 52 weeks) and (ii) physician global assessment and subject assessment (Time frame 52 weeks).

### Example 10

### Phase I Clinical Trial Evaluating Effect of a PI-3 Kinase Inhibitor Compound on the Treatment of Pulmonary Fibrosis

This study will evaluate the safety of the administration of a PI-3 kinase inhibitor for patients with idiopathic pulmonary fibrosis that have failed previous treatment.

Study Type: Interventional

Study Design: Treatment, Non-Randomized, Open Label, Uncontrolled, Single Group Assignment, Safety/Efficacy Study

Each patient is administered a twice daily dose of a compound of Formula IA, IB, IIA or IIB.

Eligibility: 35 Years to 80 Years; Both Genders Eligible for Study; Healthy Volunteers: Not accepted.

Inclusion Criteria: Diagnosis of idiopathic pulmonary fibrosis; Disease progression despite six months of treatment (steroids with/without azathioprine or cyclophosphamide) defined by at least one of the following: Increased symptoms, Decline in forced vital capacity of at least 10%, Decline in diffusion capacity for carbon monoxide of at least 20%, Increased infiltrate on CXR or high resolution CT scan, Taking <15 mg prednisone for at least 30 days prior to screening

Exclusion Criteria: Significant environmental exposure, Diagnosis of collagen vascular disease, Evidence of active infection, Clinically significant cardiac disease, Myocardial infarction, coronary artery bypass or angioplasty within 6mo, Unstable angina pectoris, Congestive heart failure requiring hospitalization within 6 months, Uncontrolled arrhythmia, Poorly controlled or severe diabetes mellitus, Pregnancy or lactation, Current enrollment in another experimental protocol

Physiologic Criteria: FEV1/FVC<0.60

Laboratory Criteria: Total bilirubin>1.5× upper limit normal, AST or ALT>3× upper limit normal, Alkaline phosphatase>3× upper limit normal, White blood cell count<2,500/mm³, Hematocrit<30%, Platelets<100,000/mm³, Prothrombin time INR>1.5.

Primary endpoint for this study is safety

Secondary endpoints: change in pulmonary function, exercise capacity, and quality of life.

### Example 11

### Clinical trial evaluating the effect of a PI-3 Kinase Inhibitor Compound on the Treatment of Liver Fibrosis

The aim of this study is to asses whether incidence of liver fibrosis is reduced in patients following a liver transplant for hepatitis C cirrhosis. The study will also assess whether incidence of fibrosis is reduced or delayed even if the infection comes back.

Study type: Interventional

Study design: Randomized, Open-label Study to Compare the Development of Liver Fibrosis at 12 Months After Transplantation for Hepatitis C Cirrhosis. Each patient is administered a thrice daily dose of PX-866 or PX-867.

Eligibility: 18 Years to 75 Years; both genders

Inclusion criteria: Reason for transplant is end-stage liver disease due to hepatitis C cirrhosis; Patients receiving a first liver transplant from a deceased or living donor; Recipients of a liver from an HCV+, HIV+ or HBV+ donor; Transplanted for liver cancer exceeding a pre-defined size; Patients with co-existing alcoholic disease who have not been abstinent for at least 6 months.

Primary Outcome Measures: Rate of fibrosis (stage 2 or above [Ishak-Knodell FS>2])

Secondary Outcome Measures: Rate of the combined endpoint of death or graft loss or FS>2; Mean fibrosis score, Percentage of patients with an increase of at least 1 stage in fibrosis; Incidence of fibrosing cholestatic hepatitis

### Example 12

### Clinical Trial Evaluating the Effect of a PI-3 Kinase Inhibitor Compound on the Treatment of Renal Fibrosis

To determine the incidence and the degree of interstitialfibrosis and arteriosclerosis, and glomerular volume in protocol biopsies at 6 months in PX-866-treated renal allograft recipients.

Study Type: Interventional

Study Design: Randomized, open label, parallel assignment, active control. Each patient is administered a thrice daily dose of PX-866 or PX-867.

Eligibility: >18 years of age

Inclusion Criteria: For renal allografts from living donors, at least one HLA-mismatch is required; Written informed consent, compliant with local regulations.

Exclusion Criteria: Recipients of a second or third renal allograft, with a past history of graft failure due to rejection; Recipients of a renal allograft from a haplotype-identical living donor or a non-heart beating donor.

Primary Outcome Measures: Primary end-point of this study will be the cortical fractional interstitial fibrosis volume in protocol biopsies at 6 months.

Secondary Outcome Measures: Patient and graft-survival at one year; serum creatinine and the estimated creatinine clearance at 6 and 12 months; intimal area and arterial wall thickness and glomerular volume in protocol biopsies at 6 months; incidence of acute rejection episodes during the first year; incidence of treatment failure

Although the present invention has been described in considerable detail with reference to certain preferred embodiments thereof, other versions are possible. Therefore the spirit and scope of the appended claims should not be limited to the description and the preferred versions contained within this specification.

1. A method of treating mild or moderate or severe pulmonary fibrosis comprising administration of a PI-3 kinase inhibitor to an individual in need thereof.

2. (canceled)

3. (canceled)

4. The method of claim 1, wherein the PI-3 kinase inhibitor is a reversible inhibitor of a PI-3 kinase.

5. The method of claim 1, wherein the PI-3 kinase inhibitor is an irreversible inhibitor of a PI-3 kinase.

6. The method of claim 1, wherein the pulmonary fibrosis is idiopathic pulmonary fibrosis.

7. The method of claim 1, wherein said pulmonary fibrosis is associated with asbestosis, cystic fibrosis, infection, exposure to environmental allergens, lung transplant, autoimmune disease, or said pulmonary fibrosis is drug-induced pulmonary fibrosis.

8. (canceled)

9. (canceled)

10. (canceled)

11. (canceled)

12. (canceled)

13. (canceled)

14. A method of treating a fibrosing syndrome in an individual diagnosed with or suspected of having a fibrosing syndrome comprising administering to the individual in need thereof a therapeutically effective amount of wortmannin or a wortmannin analogue.

15. The method of claim 14, wherein the wortmannin analogue is a compound of formula: 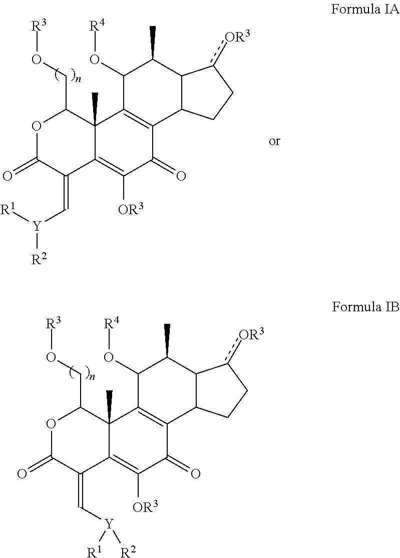
wherein:
- is an optional bond;
n is 1-6;
Y is a heteroatom
R¹ and R² are independently selected from an unsaturated alkyl, non-linear alkyl, cyclic alkyl, and substituted alkyl or R¹ and R² together with the atom to which they are attached form a heterocycloalkyl group;
R³ is absent, H, or C₁-C₆ substituted or unsubstituted alkyl;
R⁴ is (C═O)R⁵, (C═O)OR⁵, (S═O)R⁵, (SO₂)R⁵, (PO₃)R⁵, (C═O)NR⁵R⁶;
R⁵ is substituted or unsubstituted C₁-C₆ alkyl; and
R⁶ is substituted or unsubstituted C₁-C₆ alkyl.

16. The method of claim 14, wherein the compound of Formula IA or Formula IB is selected from: 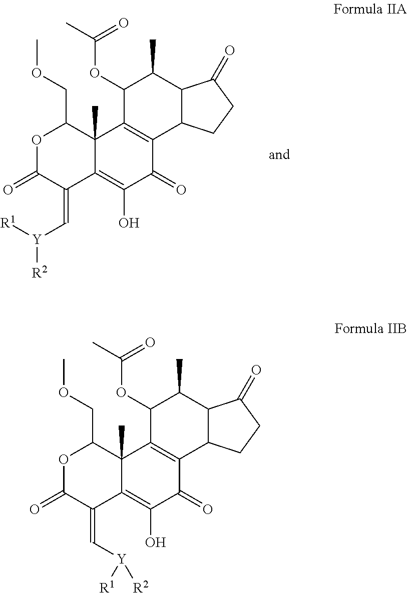
wherein Y is a heteroatom and R¹ and R² are independently selected from an unsaturated alkyl, non-linear alkyl, cyclic alkyl, and substituted alkyl.

17. (canceled)

18. (canceled)

19. The method of claim 14, wherein the wortmannin analog is a PI-3 kinase inhibitor.

20. The method of claim 19, wherein said PI-3 kinase inhibitor is PX-866.

21. The method of claim 19, wherein said PI-3 kinase inhibitor is PX-867.

22. The method of claim 14, wherein the fibrosing syndrome is mild, moderate or severe pulmonary fibrosis, cystic fibrosis, ocular fibrosis, endomyocardial fibrosis, mediastinal fibrosis, myelofibrosis, osteofibrosis, fibrosing colonoapathy, retroperitoneal fibrosis, interstitial pneumonia, progressive massive fibrosis in lungs, keloids, scleroderma, hypertrophic scarring, renal fibrosis, intestinal fibrosis, liver fibrosis, fibrosing cholestatic hepatitis, nephrogenic systemic fibrosis, fibrosis associated with organ transplantation, multifocal fibrosclerosis, or anaphylactic shock fibrosis.

23. The method of claim 14, wherein the fibrosing syndrome is mild, moderate or severe idiopathic pulmonary fibrosis.

24. The method of claim 14, wherein the fibrosing syndrome is pulmonary fibrosis associated with asbestosis, cystic fibrosis, infection, exposure to environmental allergens, autoimmune disease, or the fibrosing syndrome is drug-induced pulmonary fibrosis.

25. The method of claim 14, wherein the fibrosing syndrome is associated with organ transplant.
